(19) United States
(12) Patent Application Publication
(10) Pub. No.: US 2004/0019318 A1
(43) Pub. Date: Jan. 29, 2004

(54) Ultrasound assembly for use with a catheter

(76) Inventors: Richard R. Wilson, Seattle, WA (US); Tim Abrahamson, Seattle, WA (US); Leonard R. Oliver, Seattle, WA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/291,891

(22) Filed: Nov. 7, 2002

Related U.S. Application Data
(60) Provisional application No. 60/336,784, filed on Dec. 3, 2001.
(60) Provisional application No. 60/336,774, filed on Nov. 7, 2001.

(51) Int. Cl. A61B 17/20

(52) U.S. Cl. 604/22

(57) ABSTRACT

A catheter for delivering ultrasonic energy and therapeutic compounds to a patient's vascular system comprises an elongate outer sheath having an energy delivery section. The catheter further comprises an elongate inner core configured to be inserted into the elongate outer sheath. The elongate inner core is positioned at least partially within the energy delivery section. The catheter further comprises a plurality of ultrasound radiating members mounted along the portion of the elongate inner core within the energy delivery section. Each of the ultrasound radiating members is spaced longitudinally from the other ultrasound radiating members. The catheter further comprises an elongate drug lumen configured to deliver a therapeutic compound to the portion of the patient's vascular system adjacent to the energy delivery section.

### Priority Application

This application claims priority under 35 U.S.C. § 119(e) from U.S. Provisional Patent Application Serial No. 60/336,784, entitled “Catheter with Ultrasound Elements Attached Circumferentially” and filed Dec. 3, 2001, as well as U.S. Provisional Patent Application Serial No. 60/336,774, entitled “Ultrasound Assembly for Use with a Catheter” and filed Nov. 7, 2001, both of which are hereby incorporated by reference herein in their entirety.

### BACKGROUND OF THE INVENTION

1. Field of the Invention

The present invention relates generally to a catheter having an ultrasonic assembly, and relates specifically to a catheter configured to use ultrasonic energy to enhance the therapeutic effect of a therapeutic compound at a treatment site within the body.

2. Description of the Related Art

Several therapeutic and diagnostic applications use ultrasonic energy. For example, ultrasonic energy can be used to enhance the delivery and therapeutic effect of various therapeutic compounds. See, for example, U.S. Pat. Nos. 4,821,740, 4,953,565 and 5,007,438. In some applications, it is desirable to use an ultrasonic catheter to deliver the ultrasonic energy and/or therapeutic compound to a treatment site in the body. Such an ultrasonic catheter typically includes an ultrasonic assembly for generating the ultrasonic energy. The ultrasonic catheter can also include a delivery lumen for delivering the therapeutic compound to the treatment site. In this manner, the ultrasonic energy can be used at the treatment site to enhance the therapeutic effect and/or delivery of the therapeutic compound.

Ultrasonic catheters have successfully been used to treat human blood vessels that have become occluded or completely blocked by plaque, thrombi, emboli or other substances that reduce the blood carrying capacity of the vessel. See, for example, U.S. Pat. No. 6,001,069. To remove the blockage, the ultrasonic catheter is used to deliver solutions containing dissolution compounds directly to the blockage site. The ultrasonic energy generated by the catheter enhances the therapeutic effects of the dissolution compounds.

Ultrasonic catheters can also be used to perform gene therapy on an isolated region of a body lumen. For example, as disclosed in U.S. Pat. No. 6,135,976, an ultrasonic catheter can be provided with one or more expandable sections for occluding a section of the body lumen. A gene therapy composition is delivered to the occluded section through the delivery lumen of the catheter. The ultrasonic assembly delivers ultrasonic energy to the occluded section to enhance the entry of the gene composition into the cells of the occluded section. Other uses for ultrasonic catheters include delivering and activating light activated drugs (see, for example, U.S. Pat. No. 6,176,842).

### SUMMARY OF THE INVENTION

In certain medical procedures, it is desirable to provide an ultrasonic catheter wherein ultrasonic energy can be emitted along a circumferential region of tissue at a treatment site. In other medical procedures, it may be desirable to emit ultrasonic energy along selected angular ranges without adjusting or rotating the catheter. It may also be desirable to provide an ultrasonic catheter wherein each of the ultrasound elements is individually controllable. Finally, for ease of manufacturing and reduced costs, it may be desirable to provide a catheter on which flat ultrasound elements are mounted along the circumference of a central wire having at least three flat surfaces. The present invention addresses these needs.

According to one embodiment of the present invention, a catheter for delivering ultrasonic energy and therapeutic compounds to a patient's vascular system comprises an elongate outer sheath having an energy delivery section. The catheter further comprises an elongate inner core configured to be inserted into the elongate outer sheath. The elongate inner core is positioned at least partially within the energy delivery section. The catheter further comprises a plurality of ultrasound radiating members mounted along the portion of the elongate inner core within the energy delivery section. Each of the ultrasound radiating members is spaced longitudinally from the other ultrasound radiating members. The catheter further comprises an elongate drug lumen configured to deliver a therapeutic compound to the portion of the patient's vascular system adjacent to the energy delivery section.

According to another embodiment of the present invention, an apparatus comprises a hollow outer sheath configured to be positioned within a patient's vascular system. The apparatus further comprises an inner core configured to be received into the hollow outer sheath. The apparatus further comprises a plurality of ultrasound radiating members mounted along the inner core. Each of the ultrasound radiating members is spaced longitudinally from the other ultrasound radiating members. The apparatus further comprises a drug lumen configured to deliver a therapeutic compound to the portion of the patient's vascular system adjacent to at least one of the ultrasound radiating members.

According to another embodiment of the present invention, a method comprises positioning an elongate catheter adjacent to a treatment site within a patient's vascular system. The elongate catheter has a drug delivery lumen and an elongate inner core. The elongate inner core comprises a plurality of ultrasound radiating members spaced longitudinally thereon. The method further comprises moving the elongate inner core within the catheter such that at least one of the ultrasound radiating members is adjacent to the treatment site the method further comprises delivering ultrasonic energy from at least one of the ultrasound radiating members to the treatment site. The method further comprises delivering a therapeutic compound from the drug delivery lumen to the treatment site.

### BRIEF DESCRIPTION OF THE DRAWINGS

FIG. 1A is side view of one embodiment of an ultrasonic catheter configured for treatment of long segment peripheral arterial occlusions.

FIG. 1B is a side view of an inner core of the ultrasonic catheter of FIG. 1A.

FIG. 1C is a side view of an ultrasonic catheter comprising a drug delivery member positioned outside an internal support member.

FIG. 2A is a cross-sectional view of a distal end of the ultrasonic catheter of FIG. 1A.

FIG. 2B is a cross-sectional view of a proximal end of the ultrasonic catheter of FIG. 1A.

FIG. 2C is a cross-sectional view of an ultrasonic catheter configured to circulate a cooling fluid within a central lumen.

FIG. 2D is a cross-sectional view of an ultrasonic catheter configured to circulate a cooling fluid from a cooling lumen to a central lumen.

FIG. 2E is a cross-sectional view of an ultrasonic catheter configured to circulate a cooling fluid from a central lumen to a cooling lumen.

FIG. 3A is a side view of the distal end of the ultrasonic catheter of FIG. 1A.

FIG. 3B is a cross-sectional view of the distal end of the ultrasonic catheter of FIG. 3A.

FIG. 3C is a side view of the distal end of an ultrasonic catheter comprising a plurality of substantially linear drug delivery members.

FIG. 3D is a cross-sectional view of the distal end of the ultrasonic catheter of FIG. 3C.

FIG. 3E is a side view of the distal end of an ultrasonic catheter comprising slit-shaped drug delivery ports.

FIG. 3F is a side view of the distal end of an ultrasonic catheter comprising arcuate-shaped drug delivery ports.

FIG. 4A is a side view of the distal end of an ultrasonic catheter comprising drug delivery ports of increasing size.

FIG. 4B is a cross-sectional view of the distal end of an ultrasonic catheter comprising independent drug delivery lumens.

FIG. 5 is a cross-sectional view of the distal end of an ultrasonic catheter comprising an integral occlusion device.

FIG. 6A is a side view of the distal end of an ultrasonic catheter comprising a balloon device.

FIG. 6B is a side view of the distal end of an ultrasonic catheter comprising a balloon device and drug delivery ports of increasing size.

FIG. 6C is a side view of the distal end of an ultrasonic catheter comprising a balloon device and an expansion lumen configured to expand the balloon device and deliver a drug solution.

FIG. 6D is a side view of the distal end of an ultrasonic catheter comprising a balloon device, an expansion lumen for expanding the balloon device, and drug delivery ports of increasing size.

FIG. 7A illustrates a wiring diagram for connecting a plurality of ultrasound radiating members in parallel.

FIG. 7B illustrates a wiring diagram for connecting a plurality of ultrasound radiating members in series.

FIG. 7C illustrates a wiring diagram for connecting a plurality of ultrasound radiating members with a common wire.

FIG. 8 illustrates a wiring diagram for connecting a plurality of temperature sensors with a common wire.

FIG. 9 is a block diagram of a feedback control system for use with an ultrasonic catheter.

FIG. 10A is a side view of a treatment site.

FIG. 10B is a side view of the distal end of an ultrasonic catheter positioned at the treatment site.

FIG. 10C is a cross-sectional view of the distal end of the ultrasonic catheter of FIG. 10B positioned at the treatment site before a treatment.

FIG. 10D is a schematic diagram of the proximal end of the ultrasonic catheter of FIG. 10B.

FIG. 10E is a cross-sectional view of the distal end of the ultrasonic catheter of FIG. 10B positioned at the treatment site.

FIG. 10F is a cross-sectional view of the distal end of the ultrasonic catheter of FIG. 10B positioned at the treatment site showing movement of the inner core.

FIG. 10G is a side view of the distal end of the ultrasonic catheter of FIG. 10B positioned at the treatment site after a treatment.

FIG. 11A is a side view of an ultrasonic catheter comprising a balloon device positioned at a treatment site.

FIG. 11B is a side view of an ultrasonic catheter comprising a deployed balloon device positioned at a treatment site.

FIG. 12 is a schematic illustration of an ultrasonic catheter that is configured for insertion into small vessels of the human body.

FIG. 13A is a cross-sectional view of the distal end of the ultrasonic catheter of FIG. 12.

FIG. 13B is a cross-sectional view of the ultrasonic catheter taken through line 13B-13B of FIG. 13A.

FIG. 14A is a side view of an insulating catheter core comprising a plurality of conductive pathways electrically connected to a plurality of ultrasound assemblies.

FIGS. 14B through 14D are cross-sectional views of progressive manufacturing steps of the insulating catheter core of FIG. 14A.

FIG. 15A is a side view of an inner core comprising a piezoelectric film formed over a central conductive wire or tubing.

FIG. 15B is a cross-sectional view of the inner core of FIG. 15A illustrating electrodes formed on the piezoelectric film to create sources of ultrasonic energy.

FIG. 16A is a top view of a plurality of ultrasound radiating members mounted on a flex circuit, that is, on a ribbon containing a plurality of conductive paths.

FIG. 16B is a side view of a plurality of ultrasound radiating members mounted on a flex circuit, that is, on a ribbon containing a plurality of conductive paths.

FIG. 16C is a side view of the ribbon of FIGS. 16A and 16B wrapped around a mandrel.

FIG. 16D is a side view of a protective layer over the structure of FIG. 16C.

FIG. 17A is a top view of a plurality of ultrasound radiating members mounted on both sides of a flex circuit.

FIG. 17B is a side view of a plurality of ultrasound radiating members mounted on both sides of a flex circuit.

FIG. 17C is a side view of the ribbon of FIGS. 17A and 17B twisted to create a helical structure.

FIG. 18A is a side view of an inner core of an ultrasonic catheter, wherein the inner core has a triangular cross-section and has ultrasound elements mounted radially thereon.

FIG. 18B is a cross-sectional view of the inner core of FIG. 18A.

FIG. 19A is a side view of an inner core of an ultrasonic catheter, wherein the inner core has a rectangular cross-section and has ultrasound elements mounted radially thereon.

FIG. 19B is a cross-sectional view of the inner core of FIG. 19A.

FIG. 20 is a cross-sectional view of an inner core of an ultrasonic catheter comprising four triangular elongated members separated by an insulating material.

FIG. 21 is a cross-sectional view of an inner core of an ultrasonic catheter, wherein the elongated body is formed with a lumen.

FIG. 22 is a cross-sectional view of an inner core of an ultrasonic catheter, wherein the ultrasound radiating members are mounted radially along the exterior surface of the inner core.

FIG. 23 is a cross-sectional view of an inner core of an ultrasonic catheter, wherein flat ultrasound elements and electrical conductors are embedded into an elongated body having a square cross-section.

FIG. 24 is a cross-sectional view of an inner core of an ultrasonic catheter, wherein electrical conductors are embedded into an elongated body and flat ultrasonic elements are mounted along the exterior surfaces.

FIG. 25 is a cross-sectional view of an inner core of an ultrasonic catheter, wherein the elongated body is formed with a central lumen and an exit lumen.

### DETAILED DESCRIPTION OF PREFERRED EMBODIMENTS

Certain preferred embodiments of an ultrasonic catheter and methods of using an ultrasonic catheter are described herein. The ultrasonic catheter can be used to enhance the therapeutic effects of drugs, medication and other pharmacological agents at a treatment site within a patient's body. See, for example, U.S. Pat. Nos. 5,318,014, 5,362,309, 5,474,531, 5,628,728, 6,001,069 and 6,210,356. In one preferred embodiment, the ultrasonic catheter is adapted for use in the treatment of thrombus in the small blood vessels of the human body, such as, for example, the small cerebral arteries. In another embodiment, the ultrasonic catheter is adapted for use in the treatment of thrombus in larger blood vessels or arteries such as those located in the lower leg. However, the ultrasonic catheters disclosed herein may also find utility in other therapeutic applications, such as, for example, performing gene therapy (see, for example, U.S. Pat. No. 6,135,976), activating light activated drugs used to cause targeted tissue death (see, for example, U.S. Pat. No. 6,176,842) and causing cavitation to produce biological effects (see, for example, U.S. Pat. No. RE36,939). Moreover, such therapeutic applications may be used in various human tissues, such as, for example, other parts of the circulatory system, solid tissues, duct systems and body cavities. It is also anticipated that the ultrasonic catheters disclosed herein may find utility in other medical applications, such as, for example, diagnostic and imaging applications.

Other uses for the ultrasonic catheters and methods disclosed herein may include applications where the ultrasonic energy provides a therapeutic effect by itself, such as, for example, preventing and/or reducing stenosis and/or restenosis; tissue ablation, abrasion or disruption; promoting temporary or permanent physiological changes in intracellular or intercellular structures; or rupturing micro-balloons or micro-bubbles for drug delivery. See, for example, U.S. Pat. No. 5,269,291 and 5,431,663. The methods and apparatuses disclosed herein may also find utility in applications that do not require the use of a catheter, such as, for example, enhancing hyperthermic drug treatment; using an external ultrasonic source to enhance the therapeutic effects of drugs, medication and other pharmacological agents at a treatment site within the body; or providing a therapeutic or diagnostic effect by itself. See, for example, U.S. Pat. No. 4,821,740, 4,953,565, 5,007,438 and 6,096,000.

The entire disclosure of all of the patents mentioned in the previous two paragraphs is hereby incorporated by reference herein and made is a part of this specification.

As used herein, the term “ultrasonic energy” is a broad term and has its ordinary meaning, and further includes, without limitation, mechanical energy transferred through longitudinal pressure or compression waves with a frequency greater than about 20 kHz and less than about 20 MHz. For example, in one embodiment, the waves have a frequency between about 500 kHz and 20 MHz. In another embodiment, the waves have a frequency between about 1 MHz and 2 MHz. In yet another embodiment, the waves have a frequency of about 2 MHz.

As used herein, the term “catheter” is a broad term and has its ordinary meaning. Thus, “catheter” refers to, without limitation, a flexible tube configured to be inserted into a body cavity, duct or vessel.

As used herein, the term “therapeutic compound” refers to a drug, medicament, dissolution compound, genetic material, or any other substance capable of effecting physiological functions. Additionally, any mixture comprising any such substances is encompassed within this definition of “therapeutic compound”.

### Long Segment Ultrasonic Catheter

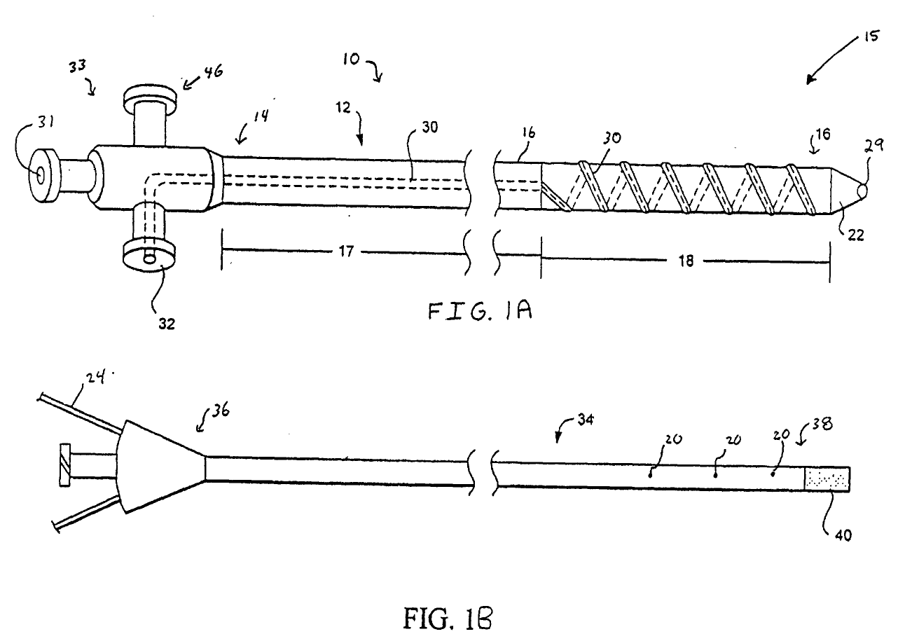

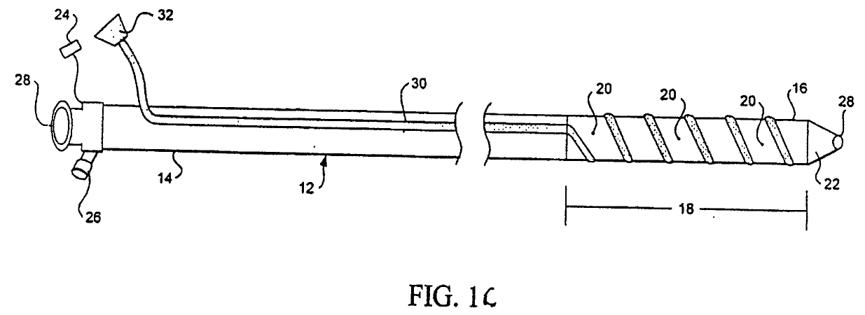

FIGS. 1A and 1B illustrate one embodiment of an ultrasonic catheter 10. Such an ultrasonic catheter 10 is configured for treatment of long segment peripheral arterial occlusions, such as those in the vascular system of the leg.

As illustrated in FIG. 1A, the ultrasonic catheter 10 generally comprises a multi-component, elongate flexible tubular body 12 having a proximal end 14 and a distal end 15. The tubular body 12 and other components of the catheter 10 can be manufactured in accordance with any of a variety of techniques well known in the catheter manufacturing field. Suitable materials and dimensions can be readily selected taking into account the natural and anatomical dimensions of the treatment site and of the desired percutaneous access site.

The tubular body 12 comprises an outer sheath 16. The outer sheath 16 preferably includes a support section 17 located at the proximal end and an energy delivery section 18 located at the distal end of the catheter 10. The support section 17 preferably comprises a material that provides the outer sheath 16 with sufficient flexibility, kink resistance, rigidity and structural support to push the energy delivery section 18 to a treatment site. Examples of such materials include, but are not limited to, extruded polytetrafluoroethylene (“PTFE”), poly-ether-ether-ketones (“PEEK”), polyethylenes (“PE”) and other similar materials. In an embodiment configured for treating thrombus in the arteries of the leg, the outer sheath 16 has an outside diameter of approximately 0.060 inches to 0.075 inches. In such an embodiment, the outer sheath 16 has an axial length of approximately 90 centimeters.

The energy delivery section 18 of the outer sheath 16 preferably comprises a material that is thinner than the material comprising the support section 17. Thinner materials generally have greater acoustic transparency than thicker materials. Suitable materials for the energy delivery section 18 include, but are not limited to, high or low density polyethylenes, urethanes, nylons, and so forth.

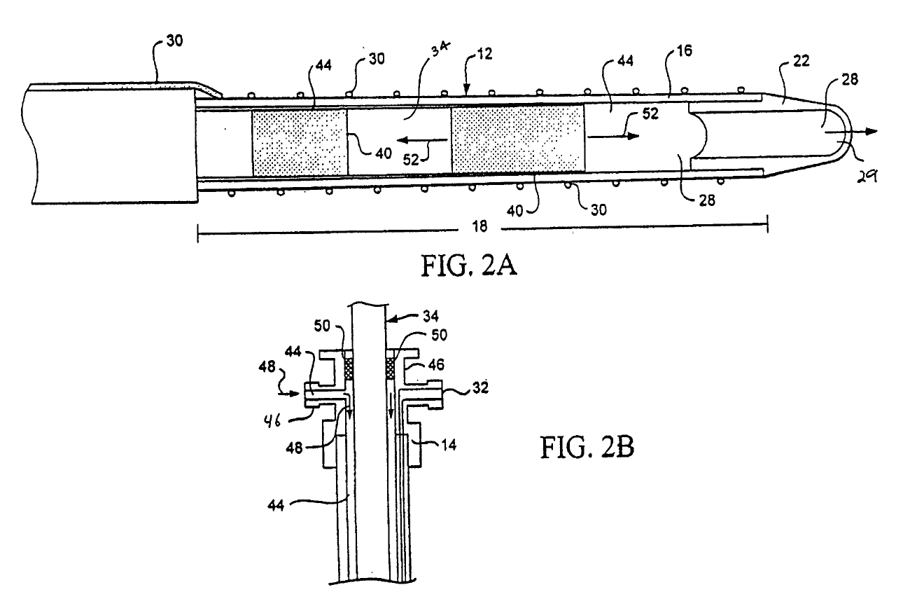

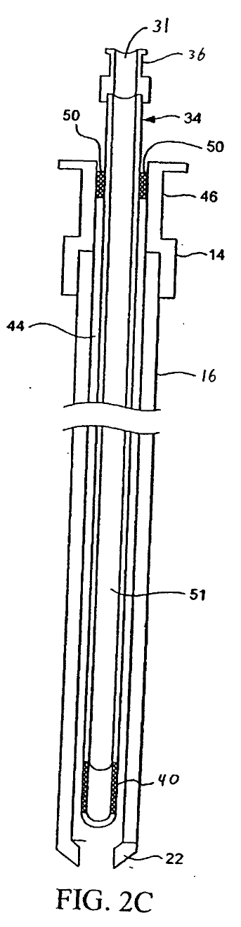

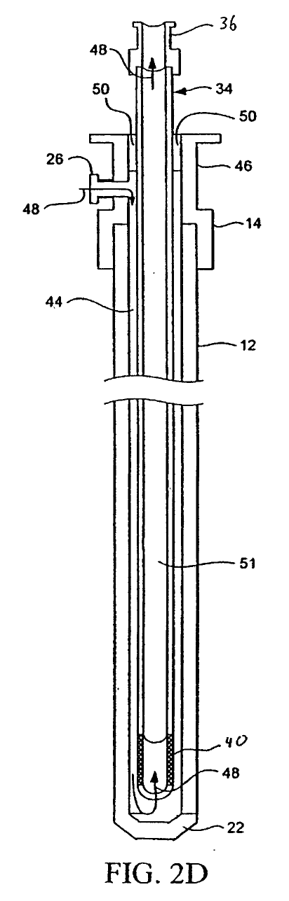

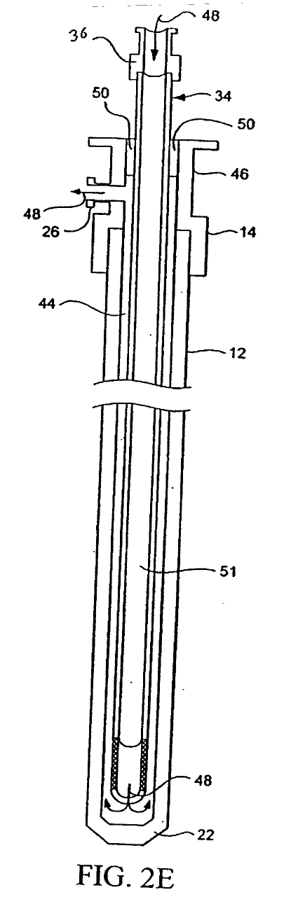

Referring now to FIGS. 1A and 2A, the outer sheath 16 defines a utility lumen 28, which preferably extends through the length of the catheter 10. As illustrated in FIG. 1A, the utility lumen 28 has a distal exit port 29 and a proximal access port 31. The proximal access port 31 forms part of backend hub 33, which is attached to the proximal end 14 of the outer sheath 16.

With continued reference to FIG. 1A, a drug delivery member 30 is positioned within the energy delivery section 18. The drug delivery member 30 comprises a drug inlet port 32, which can form part of the backend hub 33 and which can be hydraulically coupled to a drug source via a hub such as a Luer fitting. In certain embodiments, the drug delivery member 30 is incorporated into the support section 17 as illustrated in FIG. 1A. In other embodiments, the drug delivery member is external to the support section 17, as illustrated in FIG. 1C.

In certain embodiments, the catheter 10 further comprises an elongated inner core 34 comprising a proximal end 36 and a distal end 38 (see FIG. 1B). In certain embodiments, one ore more ultrasound radiating members 40 are positioned at the inner core distal end 38. The inner core 34 preferably has an outer diameter which permits the inner core 34 to be inserted into the utility lumen 28 via the proximal access port 31. FIG. 2A illustrates the inner core 34 positioned within the utility lumen 28 such that the ultrasound radiating member 40 is positioned within the energy delivery section 18. Suitable outer diameters of the inner core 34 include, but are not limited to, approximately 0.010 inches to 0.100 inches. Suitable diameters of the utility lumen 28 include, but are not limited to, approximately 0.015 inches to 0.110 inches.

In such embodiments, the ultrasound radiating member 40 can be rotated or moved within the energy delivery section 18 as illustrated by arrows 52 in FIG. 2A. The movement of the ultrasound radiating member 40 within the energy delivery section 18 can be accomplished by maneuvering the proximal end 36 of the inner core 34 while holding the back end hub 33 stationary. The inner core 34 is at least partially constructed from a material that provides enough structural support to permit movement of the inner core 34 within the outer sheath 16 without kinking of the outer sheath 16. Suitable materials for the inner core 34 include, but are not limited to polymides, polyesters, polyurethanes, thermoplastic elastomers and braided polymides.

As illustrated in FIG. 2A, the outer diameter of the inner core 34 is preferably smaller than the inner diameter of the utility lumen 28, thereby creating a cooling fluid lumen 44 between the inner core 34 and the utility lumen 28. In certain embodiments, a cooling fluid flows through the cooling fluid lumen 44, past the ultrasound radiating member 40 and through the distal exit port 29. In such embodiments, cooling fluid can be supplied via a cooling fluid fitting 46 provided in the backend hub 33 shown in FIG. 1A. As will be explained below, the flow rate of the cooling fluid and the power to the ultrasound radiating member 40 can be adjusted to maintain the temperature of the ultrasound radiating member 40 within a desired range.

As illustrated in FIG. 2B, in certain embodiments, the cooling fluid flows from the cooling fluid fitting 46 through the cooling fluid lumen 44 as illustrated by arrows 48. In such embodiments, the cooling fluid fitting 46 preferably comprises a hemostasis valve 50 having an inner diameter which substantially matches the outer diameter of the inner core 34. The matched diameters reduce leaking of the cooling fluid between the cooling fluid fitting 46 and the inner core 34.

As illustrated in FIG. 2C, in certain embodiments, the ultrasound radiating member 40 comprises a hollow cylinder and the inner core 34 defines a central lumen 51 that extends through the ultrasound radiating member 40. In such embodiments, the cooling fluid preferably flows through the central lumen and past and through the ultrasound radiating member 40, thereby cooling the ultrasound radiating member 40. In this configuration, the cooling fluid can be supplied via the proximal access port 31, with the cooling fluid fitting 46 and hemostasis valve 50 providing a seal between the inner core 34 and the outer sheath 16.

Referring again to FIG. 1A, the illustrated catheter 10 further comprises an occlusion device 22 positioned at the distal end 15 of the catheter 10. The utility lumen 28 preferably extends through the occlusion device 22. The portion of the utility lumen 28 extending through the occlusion device 22 has a diameter that can accommodate a guidewire (not shown), but that preferably prevents the ultrasound radiating member 40 from passing through the occlusion device 22. Suitable inner diameters for the occlusion device 22 include, but are not limited to, approximately 0.005 inches to 0.050 inches.

Referring now to FIG. 2D, in certain embodiments, the occlusion device 22 can be formed integrally with the sheath 16 and can have a closed end. In such embodiments, the central lumen 51 can serve as a return lumen for the cooling fluid. Consequently, both the inside and the outside of the ultrasound radiating member 40 are exposed to the cooling fluid, thereby accelerating the cooling of the ultrasound radiating member 40. As illustrated in FIG. 2E, in other embodiments, the flow of the cooling fluid can be reversed so the cooling fluid lumen 44 serves as the return cooling fluid lumen. These and other cooling fluid flow configurations permit the power provided to the ultrasound radiating member 40 to be increased in proportion to the cooling flow rate. Additionally, certain cooling fluid flow configurations can reduce exposure of the patient's body to cooling fluids.

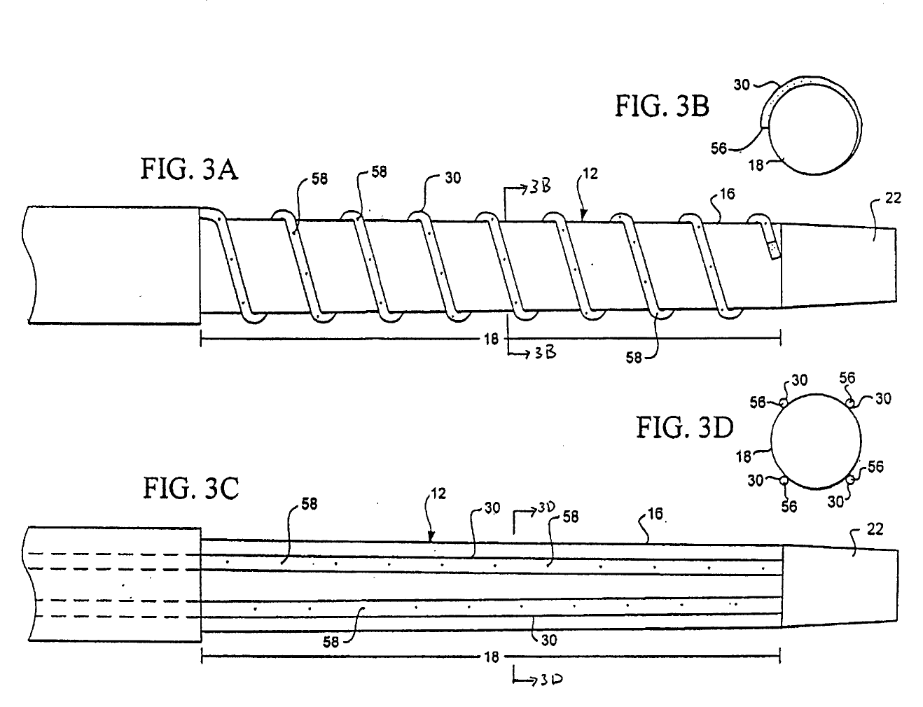

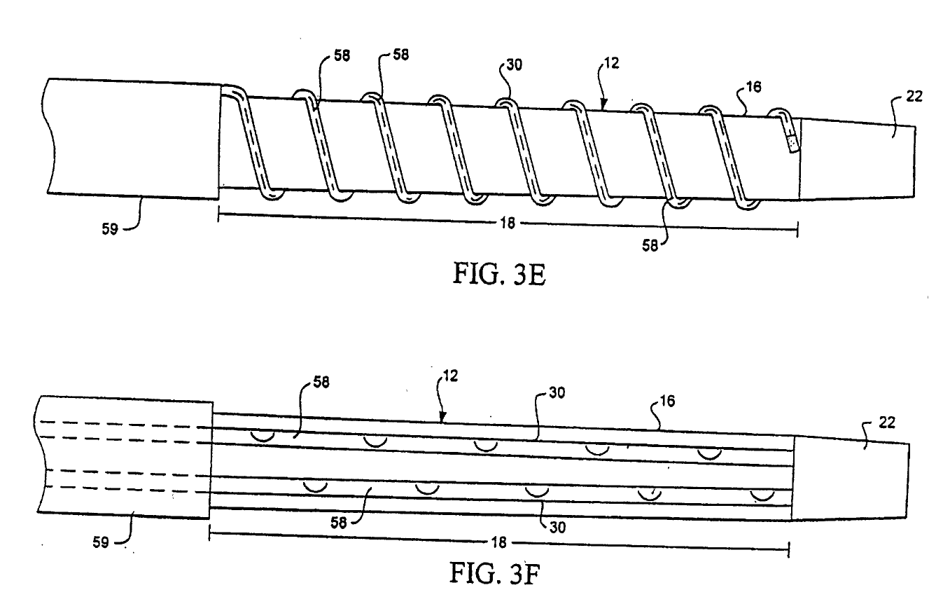

In the embodiment illustrated in FIG. 3A, the drug delivery member 30 comprises a drug delivery portion which is positioned at least partially within the energy delivery section 18. As illustrated in FIG. 3B, the drug delivery member 30 comprises a drug delivery lumen 56 extending through the length of the drug delivery member 30. The drug delivery member 30 further comprises a series of drug delivery ports 58 hydraulically coupled with the drug delivery lumen 56. In a preferred embodiment, a drug source coupled to the drug inlet port 32 provides a hydraulic pressure which drives a therapeutic compound through the drug delivery lumen 56 and out the drug delivery ports 58. A suitable material for the drug delivery member 30 includes, but is not limited to, high or low density polyethylenes, urethanes, nylons, and so forth.

In certain embodiments, the catheter 10 includes a plurality of drug delivery members 30. The drug delivery members 30 can be wound around the energy delivery section 18 or they can be positioned along the length of the energy delivery section 18 as illustrated in FIG. 3C. Each drug delivery member 30 can be coupled to the same drug inlet port 32. In other embodiments, however, each drug delivery member 30 is coupled to an independent drug inlet port 32, thereby allowing different therapeutic compounds to be delivered to different drug delivery ports 58.

The drug delivery ports 58 are preferably positioned close enough together to achieve a substantially even flow of therapeutic compound around the circumference of the energy delivery section 18 and along the length of the energy delivery section 18. The proximity of adjacent drug delivery ports 58 can be changed by changing the linear density of drug delivery ports 58 along the drug delivery member 30, by changing the number of windings of the drug delivery member around the energy delivery section 18, or by changing the number of drug delivery members 30 included within the energy delivery section 18. In one embodiment, the displacement between adjacent drug delivery members 30 is between approximately 0.1 inches and 1.0 inches and more preferably between approximately 0.2 inches and 0.6 inches.

The size of the drug delivery ports 58 can be constant or variable along the length of the drug delivery member 30. For example, in certain embodiments, the size of distally-positioned drug delivery ports 58 is larger than the size of proximally-positioned drug delivery ports 58. The increase in size of the drug delivery ports 58 can be configured to produce similar flow rates of therapeutic compound through each drug delivery port 58. A similar flow rate from each drug delivery port 58 increases the uniformity of therapeutic compound flow rate along the length of the sheath 16. For example, in one embodiment in which the drug delivery ports 58 have similar sizes along the length of the drug delivery member, the drug delivery ports 58 have a diameter of approximately 0.0005 inches to 0.0050 inches. In another embodiment in which the size of the drug delivery ports 58 changes along the length of the drug delivery member 30, the drug delivery ports 58 have a diameter of approximately 0.0001 inches to 0.005 inches at the proximal end of the energy delivery section 18, and approximately 0.0005 inches to 0.0020 inches at the distal end of the energy delivery section 18. The increase in size between adjacent drug delivery ports can be substantially uniform between separate drug delivery members 30, or along the same drug delivery member 30. The increase in size between adjacent drug delivery ports depends on the material comprising the drug delivery member 30 and on the diameter of the drug delivery member 30. The drug delivery ports 58 can be created in the drug delivery member 30 by punching, drilling, burning (such as with a laser), or by any other suitable method.

Uniform therapeutic compound flow along the length of the sheath 16 can also be increased by increasing the density of the drug delivery ports 58 toward the distal end of the drug delivery member 30. As illustrated in FIG. 3E, in certain embodiments, the drug delivery ports 58 comprise slits having a linear shape. In other embodiments, as illustrated in FIG. 3F, the drug delivery ports 58 comprise slits having an arcuate shape. Regardless of the shape of the drug delivery portions 58, the drug delivery member 30 can comprise materials such as polyimide, nylon, Pebax®, polyurethane or silicon. In embodiments wherein the drug delivery ports 58 comprise slits, when the drug delivery lumen 56 is filled with a therapeutic compound, the slits remain closed until the hydraulic pressure within the drug delivery lumen 56 exceeds a threshold pressure. As the hydraulic pressure within the drug delivery lumen 56 builds, the pressure on each of the slits will be approximately uniform. Once the threshold pressure is reached, the plurality of drug delivery ports 58 will open substantially simultaneously and will thereby cause a nearly uniform flow of therapeutic compound from the plurality of slit-shaped drug delivery ports 58. Similarly, when the hydraulic pressure within the drug delivery lumen 56 falls below the threshold pressure, the slit-shaped drug delivery ports 58 close and prevent delivery of additional therapeutic compound. The stiffer the material used to construct the drug delivery member 30, the higher the threshold pressure required to open the slit-shaped drug delivery ports 58. The slit shape can also prevent the drug delivery ports 58 from opening when exposed to low pressures from outside the sheath 16. Consequently, slit-shaped drug delivery ports 58 can increase control of drug delivery.

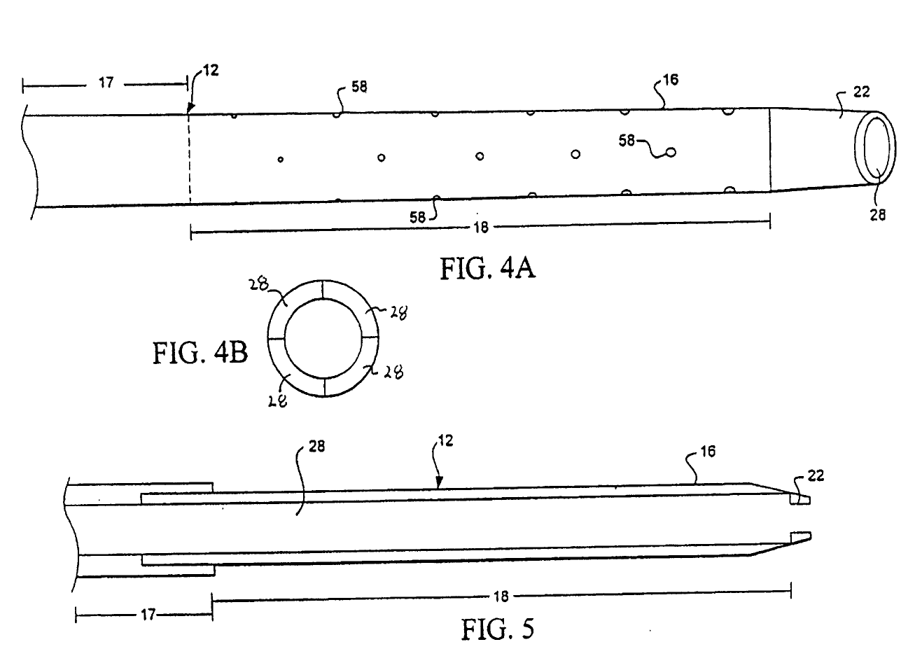

In the embodiment illustrated in FIG. 4A, the outer sheath 16 and energy delivery section 18 are constructed from a single material. Suitable materials include, but are not limited to, high or low density polyethylenes, urethanes, nylons, and so forth. Additionally, the entire outer sheath 16, or only the proximal end of the outer sheath 16, can be reinforced by braiding, mesh or other constructions configured to increase pushability. As illustrated in FIG. 4A, the drug delivery ports 58 can be included in the outer sheath 16. In such embodiments, the drug delivery ports 58 can be coupled to independent drug delivery lumens 30 formed integrally with the outer sheath 16 as illustrated in FIG. 4B.

In the embodiment illustrated in FIG. 5, the outer sheath 16 includes a support section 17 which is constructed from a different material than the energy delivery section 18. As mentioned above, the energy delivery section 18 is preferably constructed from a material that readily transmits ultrasound energy. The support section 17 is preferably constructed from a material that provides structural strength and kink resistance. Additionally, in certain embodiments, the support section 17, or the proximal end of the support section 17, is reinforced by braiding, mesh or other constructions configured to increase kink resistance and pushability. Suitable materials for the support section 17 include, but are not limited to, PTFE, PEEK, PE and other similar materials. A suitable outer diameter for the support section 17 includes, but is not limited to, approximately 0.020 inches to 0.200 inches. Suitable materials for the energy delivery section 18 include, but are not limited to, high and low density polyethylenes, urethanes, nylons, and other materials having low ultrasound impedance. Low ultrasound impedance materials are materials which readily transmit ultrasound energy with minimal absorption of the ultrasound energy. FIG. 5 also illustrates the occlusion device 22 as being integrally formed with the energy delivery section 18.

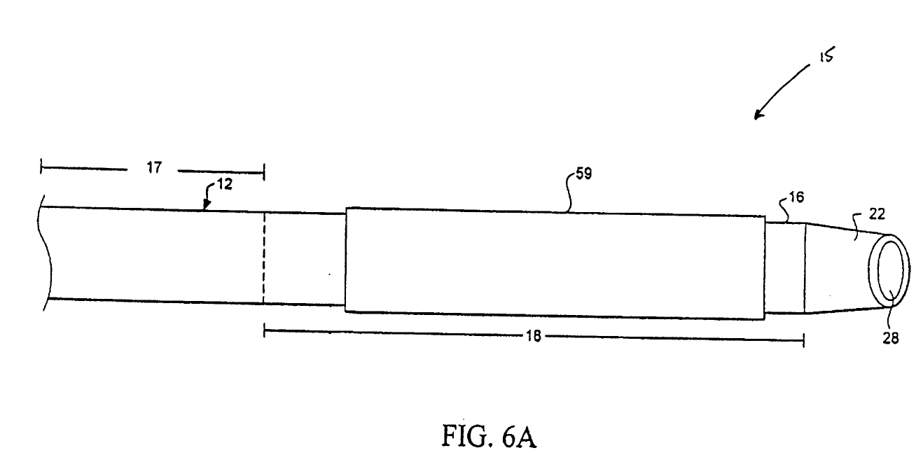

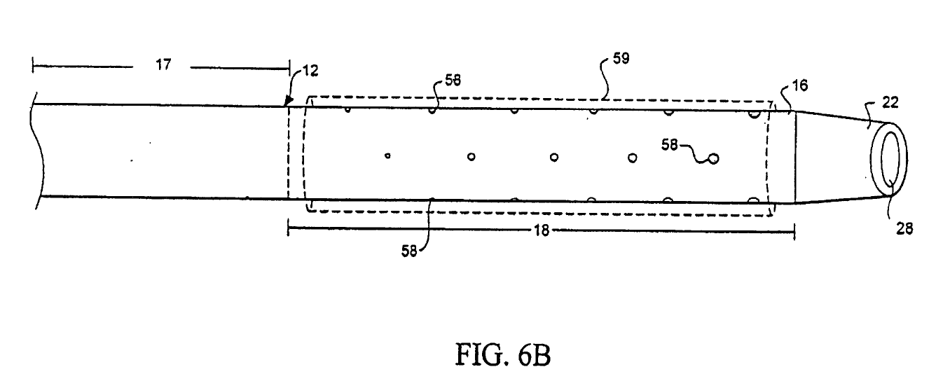

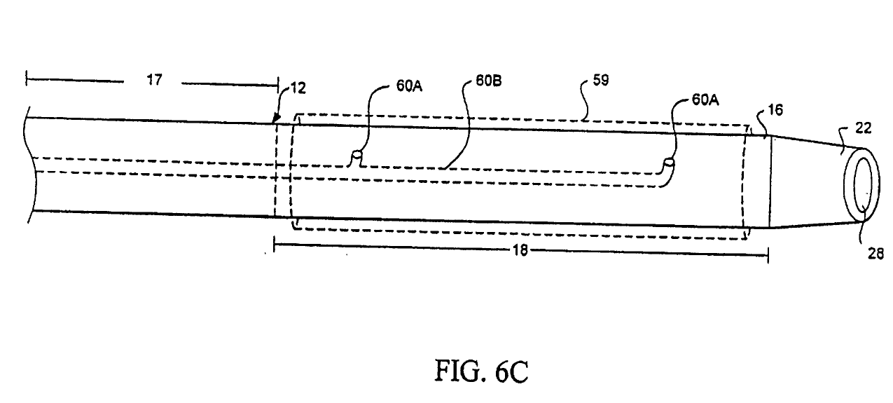

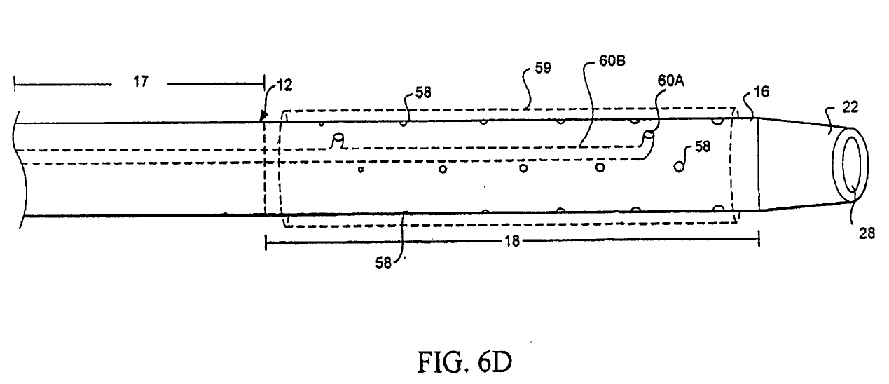

In the embodiment illustrated in FIG. 6A, the distal end 15 of the catheter 10 further comprises a balloon device 59. The balloon device 59 can be constructed from a permeable membrane or from a selectively permeable membrane which allows certain media to flow therethrough while preventing other media from flowing therethrough. Suitable materials for the balloon device 59 include, but are not limited to, cellulose, cellulose acetate, polyvinylchloride, polyolefin, polyurethane and polysulfone. When the balloon device 59 is constructed from a permeable membrane or from a selectively permeable membrane, the membrane pore sizes are preferably approximately 5Å to 2 μm, more preferably approximately 50Å to 900Å, and in yet another embodiment approximately 100 Å to 300Å in diameter.

As illustrated in FIG. 6B, in certain embodiments, the balloon device 59 is positioned adjacent drug delivery ports 58. As described above, the drug delivery ports 58 can be configured to produce a uniform flow of therapeutic compound along the length of the energy delivery section 18. This configuration can substantially prevent a pressure gradient from developing along the length of the balloon device 59. In such embodiments, delivering a therapeutic compound through the drug delivery ports 58 can cause the balloon device 59 to expand. In embodiments wherein the balloon device 59 comprises a membrane or a selectively permeable membrane, the therapeutic compound can be delivered with sufficient pressure to drive the drug across the membrane. In other embodiments, carious phoretic processes and apparatuses are used to drive the therapeutic compound across the membrane. In embodiments wherein the balloon device 59 comprises a selectively permeable membrane, the pressure and/or phoresis may drive certain components of the therapeutic compound across the membrane while other components are prevented from crossing the membrane.

As illustrated in FIG. 6C, the balloon device 59 can also be positioned adjacent one or more expansion ports 60A coupled to an expansion lumen 60B. In such embodiments, the therapeutic compound can be delivered to the balloon device 59 via the expansion lumen 60B. Delivering a therapeutic compound through the expansion lumen 60B can serve to expand the balloon device 59. When the balloon device 59 is constructed from a membrane or a selectively permeable membrane, the therapeutic compound can be delivered with sufficient pressure to drive the therapeutic compound, or certain components of the therapeutic compound, across the membrane. Similarly, phoretic means can also be used to drive the therapeutic compound, or certain components of the therapeutic compound, across the membrane.

As illustrated in FIG. 6D, the balloon device 59 can also be positioned adjacent expansion ports 60A that are coupled with both an expansion lumen 60B and drug delivery ports 58. In such embodiments, different therapeutic compounds can be delivered through the expansion ports 60B and the drug delivery ports 58. In other embodiments, a media suitable for expanding the balloon device 59 is delivered through the expansion lumen 60B and the expansion ports 60A while the drug solution is delivered through the drug delivery ports 58. In embodiments wherein the balloon device 59 is constructed from a membrane or a selectively permeable membrane, a medium that wets the membrane and enhances the permeability of the membrane can be delivered through the expansion ports 60A. In such embodiments, a therapeutic compound can be delivered through the drug delivery ports 58 concurrently with or after the wetting medium has been delivered.

In the illustrated embodiments discussed above, the ultrasound radiating member 40 comprises an ultrasonic transducer, which converts, for example, electrical energy into ultrasonic energy. A suitable example of an ultrasonic transducer for generating ultrasonic energy from electrical energy includes, but is not limited to, piezoelectric ceramic oscillators. In modified embodiments, the ultrasonic energy can be generated by an ultrasonic transducer that is remote from the ultrasound radiating member 40 and the ultrasonic energy can be transmitted via, for example, a wire that is coupled to the ultrasound radiating member 40.

In the illustrated embodiments, the ultrasound radiating member 40 comprises an ultrasonic transducer having a cylindrical shape. In other embodiments, the ultrasonic transducer can comprise a thin block. In still other embodiments, the ultrasonic transducer can comprise a hollow cylinder or a disk, either of which may or may not be concentric about the inner core 34. The ultrasound radiating member 40 can also be formed from an array of smaller ultrasound radiating members.

As described above, suitable frequencies for the ultrasound radiating member 40 include, but are not limited to, from about 20 kHz to about 20 MHz. Preferably, the frequency is between about 500 kHz and 20 MHz, and more preferably between about 1 MHz and 2 MHz. In yet another embodiment, the sound waves have a frequency of about 2 MHz.

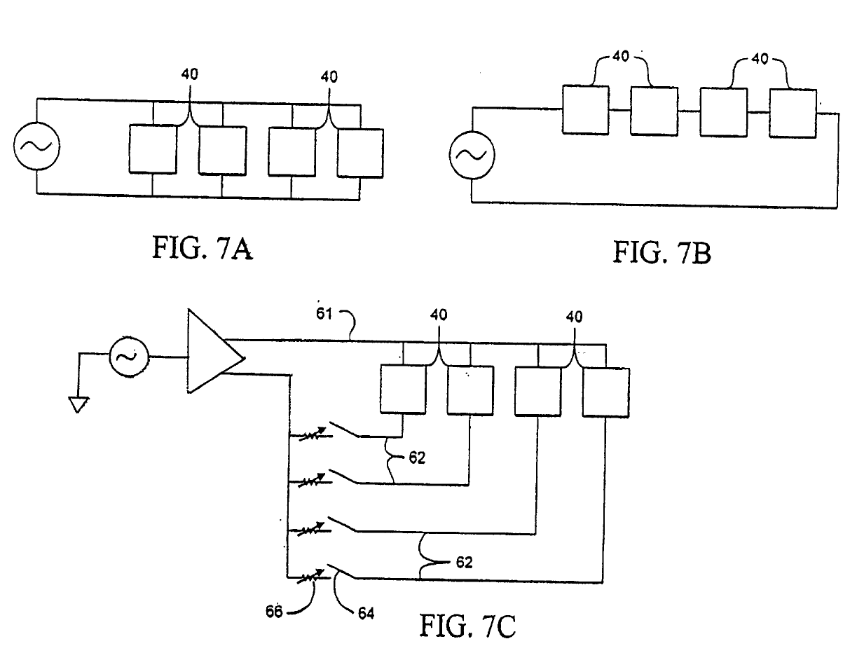

In certain embodiments, each ultrasound radiating member 40 is individually powered. In embodiments wherein the inner core 34 includes n ultrasound radiating members 40, the inner core 34 preferably includes 2n wires to individually power the n ultrasound radiating members 40. The individual ultrasound radiating members 40 can also be electrically connected in parallel (as illustrated in FIG. 7A) or in series (as illustrated in FIG. 7B). These arrangements permit more flexibility by requiring fewer wires. Each of the ultrasound radiating members 40 can receive power simultaneously, regardless whether the ultrasound radiating members 40 are connected in series or in parallel. When the ultrasound radiating members 40 are connected in series, less current is required to produce the same power from each ultrasound radiating member 40 than when the ultrasound radiating members 40 are connected in parallel. A reduced current requirement allows smaller wires to be used to provide power to the ultrasound radiating members 40 and accordingly increases the flexibility of the inner core 34. When the ultrasound radiating members 40 are connected in parallel, one ultrasound radiating member 40 can break down without affecting the current flow to the remaining ultrasound radiating members 40, which will continue to operate.

Preferably, the output power of the ultrasound radiating members 40 is controllable. For example, in the embodiment illustrated in FIG. 7C, a common wire 61 provides power to all of the ultrasound radiating members 40, each of which has an individual return wire 62. In such embodiments, a particular ultrasound radiating member 40 can be individually activated by closing a switch 64 to complete a circuit between the common wire 61 and the ultrasound radiating member's individual return wire 62. Once a switch 64 corresponding to a particular ultrasound radiating member 40 has been closed, the amount of power supplied to the ultrasound radiating member 40 can be adjusted using the potentiometer 66 corresponding to that particular ultrasound radiating member 40. Accordingly, an inner core 34 comprising n ultrasound radiating members 40 requires only n+1 wires while still permitting independent control of the ultrasound radiating members 40. A reduced number of wires within the inner core 34 increases the flexibility of the inner core 34. To further increase the flexibility of the inner core 34, the individual return wires 62 preferably have diameters which are smaller than the common wire 61 diameter. For instance, in an embodiment where n ultrasound radiating members 40 will be powered simultaneously, the diameter of the individual return wires 62 can be approximately \sqrt{n} times smaller than the diameter of the common wire 61.

In certain embodiments, as illustrated in FIG. 1B, the inner core 34 of the catheter 10 further comprises one or more temperature sensors 20, which are preferably located at the distal end 38 of the inner core 34. In such embodiments, the proximal end 36 of the inner core 34 includes a temperature sensor lead 24, which is operatively connected to the temperature sensors. In a modified embodiment, as illustrated in FIG. 1C, the temperature sensors 20 are positioned within the energy delivery section 18, outside the outer sheath 16. In such embodiments, the temperature sensor lead 24 extends from the proximal end 14 of the catheter 10. Suitable temperature sensors 20 include, but are not limited to, temperature sensing diodes, thermistors, thermocouples, resistance temperature detectors (“RTD”) and fiber optic temperature sensors which use thermalchromic liquid crystals. Suitable temperature sensor 20 geometries include, but are not limited to, a point, a patch, a stripe or a band around the outer sheath 16. The temperature sensors 20 can be positioned on the outer sheath 16 or on the inner core 34 near the ultrasound radiating members 40. The temperature sensors 20 are preferably positioned to be exposed near the energy delivery section 18.

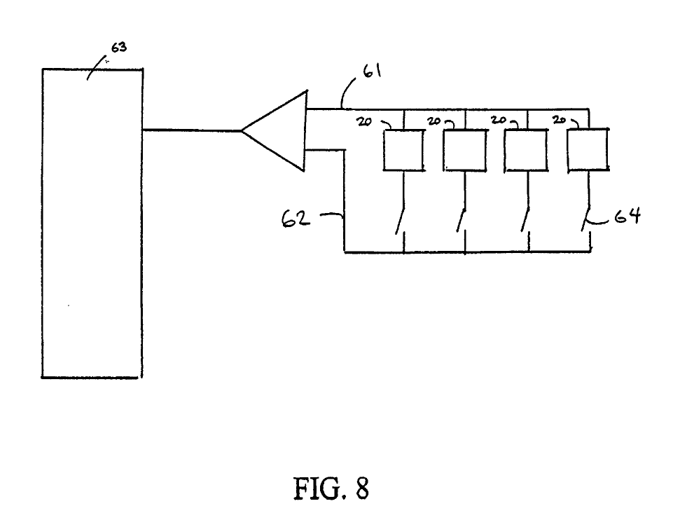

FIG. 8 illustrates one embodiment for electrically connecting the temperature sensors 20. In such embodiments, each temperature sensor 20 is coupled to a common wire 61 is associated with an individual return wire 62. Accordingly, n+1 wires can be used to independently sense the temperature at n distinct temperature sensors 20. The temperature at a particular temperature sensor 20 can be determined by closing a switch 64 to complete a circuit between that thermocouple's individual return wire 62 and the common wire 61. In embodiments wherein the temperature sensors 20 comprise thermocouples, the temperature can be calculated from the voltage in the circuit using, for example, a sensing circuit 63. To improve the flexibility of the outer sheath 16, the diameters of the individual return wires 62 preferably are smaller than the diameter of the common wire 61.

In other embodiments, each temperature sensor 20 is independently wired. In such embodiments, 2n wires pass the length of the outer sheath 16 to independently sense the temperature at n independent temperature sensors 20.

In still other embodiments, the flexibility of the outer sheath 16 and the inner core 34 can be improved by using fiber optic based temperature sensors 20. In such embodiments, flexibility can be improved because only n fiber optic members are employed to sense the temperature at n independent temperature sensors 20.

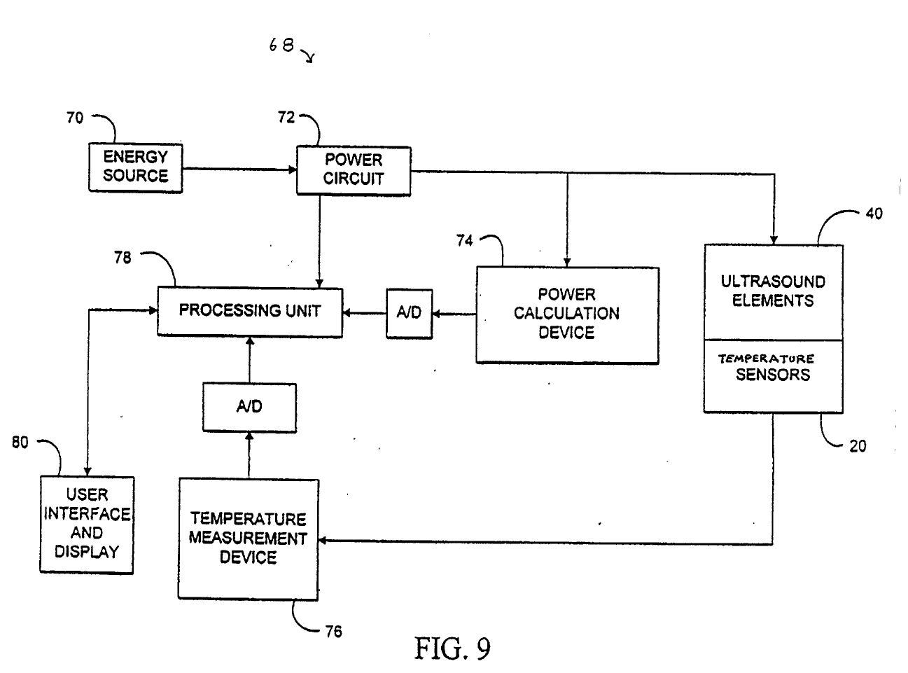

FIG. 9 illustrates one embodiment of a feedback control system 68 that can be used with the catheter 10. Such embodiments allow the temperature at each temperature sensor 20 to be monitored and allow the output power of the energy source 70 to be adjusted accordingly. A physician can, if desired, override the closed or open loop system.

The feedback control system 68 preferably comprises an energy source 70, power circuits 72 and a power calculation device 74 that is coupled to the ultrasound radiating members 40. A temperature measurement device 76 is coupled to the temperature sensors 20 on the outer sheath 16 or on the inner core 34. A processing unit 78 is coupled to the power calculation device 74, the power circuits 72 and a user interface and display 80.

In operation, the temperature at each temperature sensor 20 is determined by the temperature measurement device 76. The processing unit 78 receives each determined temperature from the temperature measurement device 76. The determined temperature can then be displayed to the user at the user interface and display 80.

The processing unit 78 comprises logic for generating a temperature control signal. The temperature control signal is proportional to the difference between the measured temperature and a desired temperature. The desired temperature can be determined by the user (at set at the user interface and display 80) or can be preset within the processing unit 78.

The temperature control signal is received by the power circuits 72. The power circuits 72 are preferably configured to adjust the power level, voltage, phase and/or current of the electrical energy supplied to the ultrasound radiating members 40 from the energy source 70. For example, when the temperature control signal is above a particular level, the power supplied to a particular ultrasound radiating member 40 is preferably reduced in response to that temperature control signal. Similarly, when the temperature control signal is below a particular level, the power supplied to a particular ultrasound radiating member 40 is preferably increased in response to that temperature control signal. After each power adjustment, the processing unit 78 preferably monitors the temperature sensors 20 and produces another temperature control signal which is received by the power circuits 72.

The processing unit 78 preferably further comprise safety control logic. The safety control logic detects when the temperature at a temperature sensor 20 has exceeded a safety threshold. The processing unit 78 can then provide a temperature control signal which causes the power circuits 72 to stop the delivery of energy from the energy source 70 to the ultrasound radiating members 40.

Because, in certain embodiments, the ultrasound radiating members 40 are mobile relative to the temperature sensors 20, it can be unclear which ultrasound radiating member 40 should have a power, voltage, phase and/or current level adjustment. Consequently, each ultrasound radiating member 40 is preferably identically adjusted. In a modified embodiment, the power, voltage, phase, and/or current supplied to each of the ultrasound radiating members 40 is adjusted in response to the temperature sensor 20 which indicates the highest temperature. Making voltage, phase and/or current adjustments in response to the temperature sensed by the temperature sensor 20 indicating the highest temperature can reduce overheating of the treatment site.

The processing unit 78 also receives a power signal from a power calculation device 74. The power signal can be used to determine the power being received by each ultrasound radiating member 40. The determined power can then be displayed to the user on the user interface and display 80.

As described above, the feedback control system 68 can be configured to maintain tissue adjacent to the ultrasound radiating members 40 below a desired temperature. For example, it is generally desirable to prevent tissue adjacent the ultrasound radiating members 40 from increasing more than 6° C. As described above, the ultrasound radiating members 40 can be electrically connected such that each ultrasound radiating member 40 generates an independent output. In certain embodiments, the output from the power circuit maintains a selected energy at each ultrasound radiating member 40 for a selected length of time.

The processing unit 78 can comprise a digital or analog controller, such as for example a computer with software. When the processing unit 78 is a computer it can include a central processing unit (“CPU”) coupled through a system bus. As is well known in the art, the user interface and display 80 can comprise a mouse, a keyboard, a disk drive, a display monitor, a nonvolatile memory system, or any another. Also preferably coupled to the bus is a program memory and a data memory.

In lieu of the series of power adjustments described above, a profile of the power to be delivered to each ultrasound radiating member 40 can be incorporated into the processing unit 78, such that a preset amount of ultrasonic energy to be delivered is preprofiled. In such embodiments, the power delivered to each ultrasound radiating member 40 can then be adjusted according to the preset profiles.

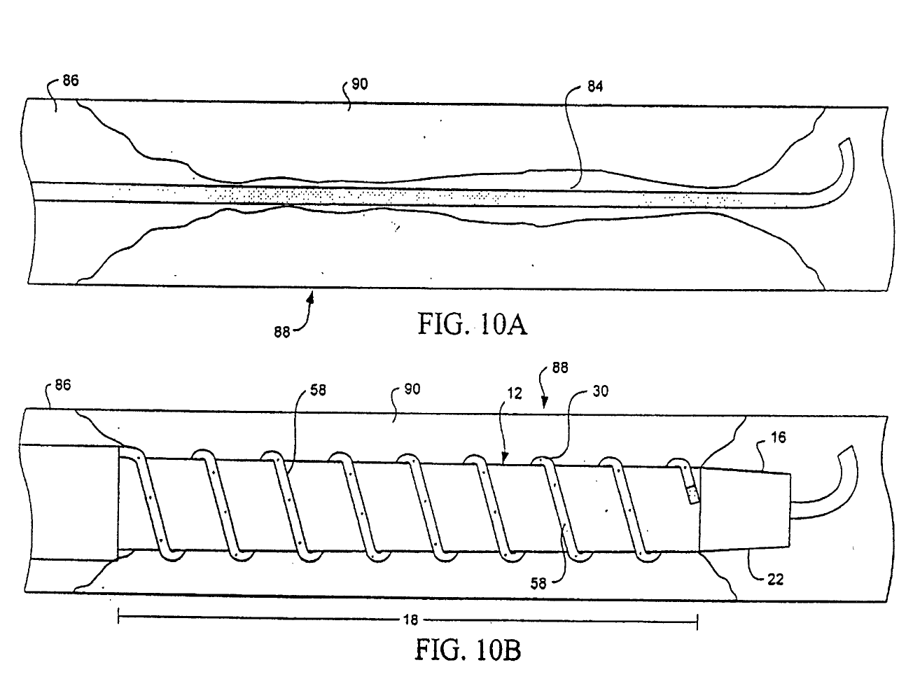

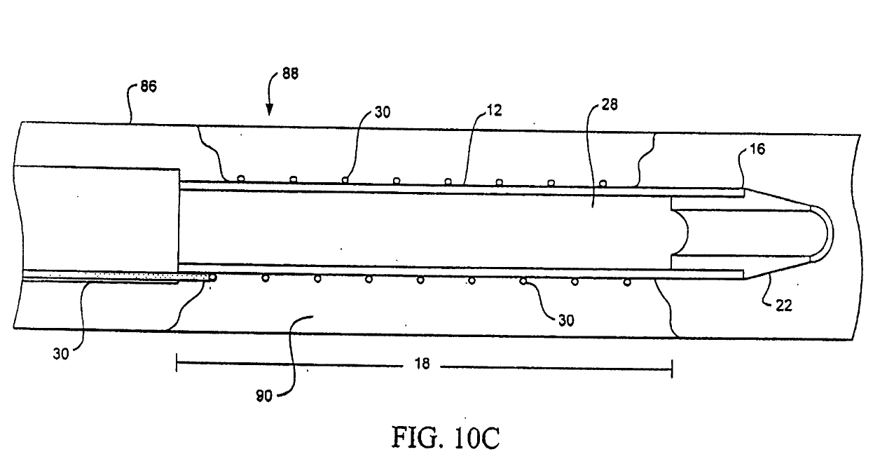

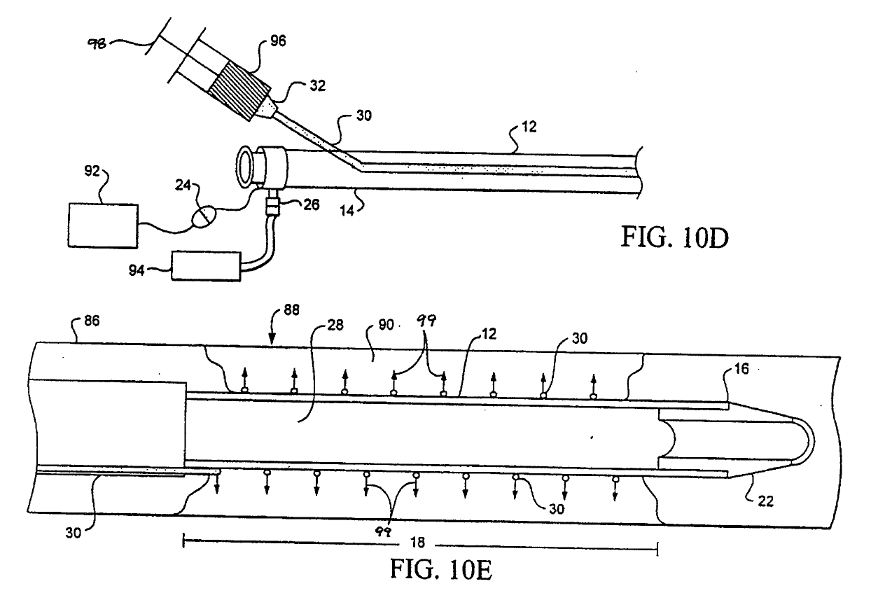

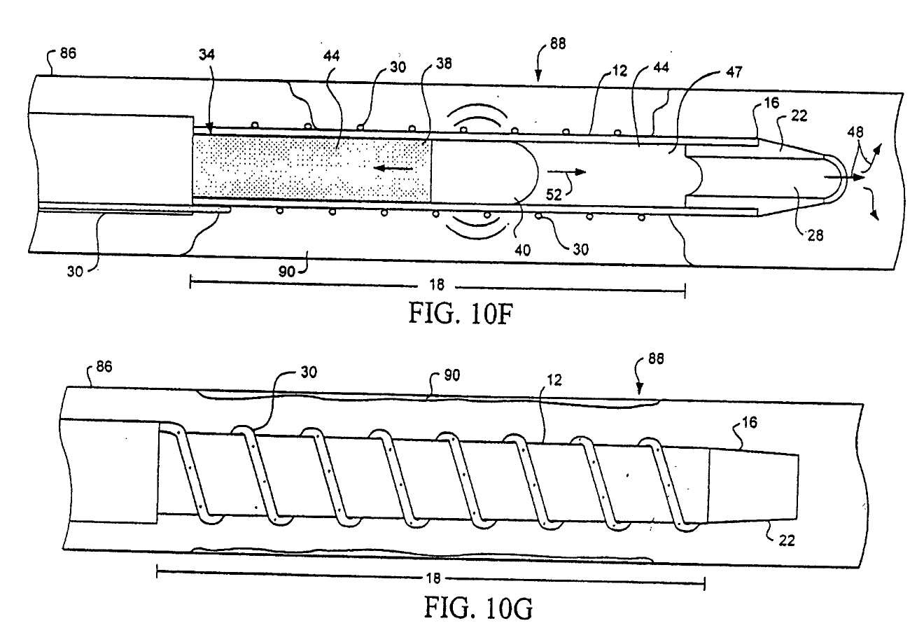

FIGS. 10A through 10G illustrate a method for using the ultrasonic catheter 10. As illustrated in FIG. 10A, a guidewire 84 similar to a guidewire used in typical angioplasty procedures is directed through a patient's vessels 86 to a treatment site 88 which includes a clot 90. The guidewire 84 is directed through the clot 90. Suitable vessels 86 include, but are not limited to, the large periphery blood vessels of the body. Additionally, as mentioned above, the ultrasonic catheter 10 also has utility in various imaging applications or in applications for treating and/or diagnosing other diseases in other body parts.

As illustrated in FIG. 10B, the utility lumen 28 of the outer sheath 16 is slid over the guidewire 84, and the outer sheath 16 is advanced along the guidewire 84 using conventional over-the-guidewire techniques. The outer sheath 16 is advanced until the energy delivery section 18 of the outer sheath 16 is positioned at the clot 90. In certain embodiments, radiopaque markers (not shown) are positioned along the energy delivery section 18 of the outer sheath 16 to aid in the positioning of the outer sheath 16 within the treatment site 88.

As illustrated in FIG. 10C, the guidewire 84 is withdrawn from the utility lumen 28 by pulling the guidewire 84 at the proximal end 14 of the catheter 10 while holding the outer sheath 16 stationary. As illustrated in FIG. 10D, a temperature monitor 92 is coupled to the temperature sensor lead 24; a cooling fluid source 94 is coupled to the cooling fluid fitting 46; and a therapeutic compound source 96 is coupled to the drug inlet port 32. In certain embodiments, the therapeutic compound source 96 comprises a syringe with a Luer fitting which is complementary with the drug inlet port 32. In such embodiments, pressure applied to a plunger 98 on the therapeutic compound source 96 drives the therapeutic compound through the drug delivery lumen 56. The therapeutic compound is delivered from the drug delivery lumen 56 through the drug delivery ports 58 as illustrated by the arrows 99 in FIG. 10E. Suitable therapeutic compounds include, but are not limited to, an aqueous solution containing Heparin, Uronkinase, Streptokinase and Tissue Plasminogen Activator (“TPA”).

As illustrated in FIG. 10F, the inner core 34 is inserted into the utility lumen 28 until the ultrasound radiating member 40 is positioned at least partially within the energy delivery section 18. To aid in placement of the ultrasound radiating member 40 within the energy delivery section 18, radiopaque markers (not shown) can be positioned on the inner core 34 adjacent to each of the ultrasound radiating members 40, or the ultrasound radiating members 40 themselves can be radiopaque. In other embodiments, the ultrasound energy radiated by the ultrasound radiating members 40 can be used to aid placement. Once the inner core 34 is properly positioned, the ultrasound radiating member 40 is activated to deliver ultrasonic energy through the energy delivery section 18 to the clot 90. Suitable ultrasonic energy is delivered with a frequency from about 20 kHz to 20 MHz. More preferably, the ultrasonic energy is delivered with a frequency from about 500 kHz to 20 MHz. Even more preferably, the ultrasonic energy is delivered with a frequency from about 1 MHz to 2 MHz. In yet another embodiment, the ultrasonic energy is delivered with a frequency of about 2 MHz. While the ultrasonic energy is being delivered, the ultrasound radiating member 40 can be moved within the energy delivery section 18 as illustrated by the arrows 52. The movement of the ultrasound radiating member 40 within the energy delivery section 18 can be caused by manipulating the proximal end 36 of the inner core 34 while holding the backend hub 33 stationary. In the embodiment illustrated in FIG. 10F, arrows 48 indicated that a cooling fluid flows through the cooling fluid lumen 44 and out the occlusion device 22.

The cooling fluid can be delivered before, after, during or intermittently with the delivery of ultrasonic energy. Similarly, the therapeutic compound can be delivered before, after, during or intermittently with the delivery of ultrasonic energy. Consequently, the steps illustrated in FIGS. 10A through 10F can be performed in a variety of different orders than that described above. The therapeutic compound and energy are preferably applied until the clot 90 is partially or entirely dissolved, as illustrated in FIG. 10G. Once the clot 90 has been dissolved to the desired degree, the outer sheath 16 and the inner core 34 are withdrawn from the treatment site 88.

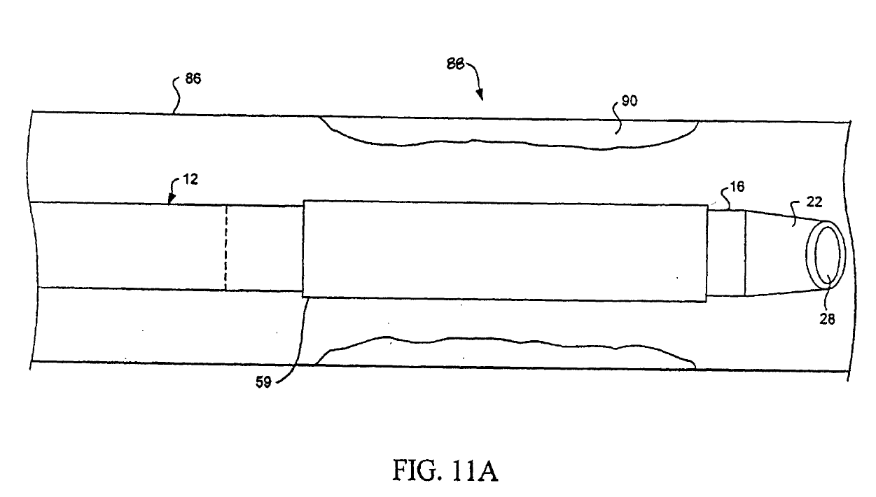

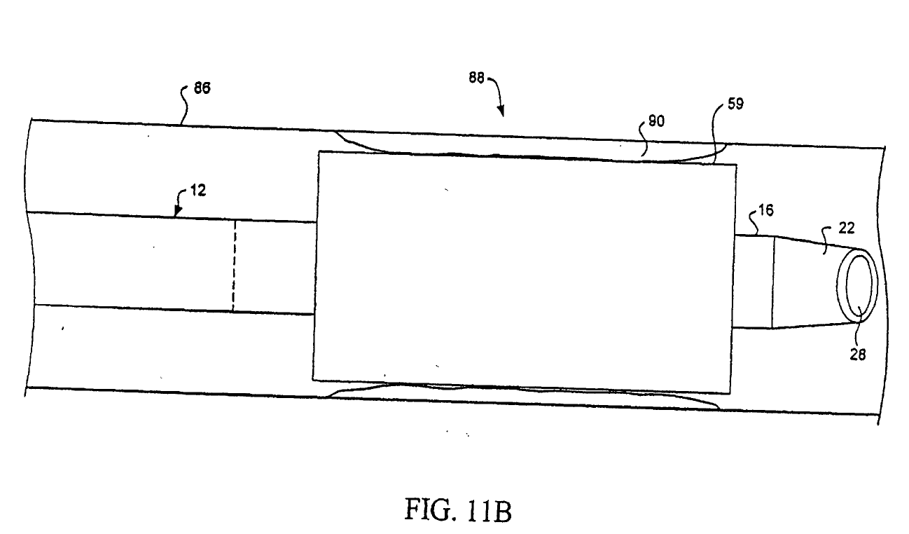

FIGS. 11A and 11B illustrate a method for using the catheter 10 when the distal end 15 of the catheter 10 includes a balloon device 59. In such embodiments, the catheter 10 is advanced through a vessel 86, as described above, until the balloon device 59 is positioned adjacent to the treatment site 88, as illustrated in FIG. 11A. The balloon device 59 is expanded until the balloon device 59 contacts the clot 90 as illustrated in FIG. 11B. As described above, the balloon device 59 can be expanded by delivering a therapeutic compound through an expansion port 60A or a drug delivery port 58. Or, the balloon device 59 can be expanded by delivering an expansion media through an expansion port 60A. Once the balloon device 59 contacts the clot 90, the therapeutic compound or components of the therapeutic compound are driven across the membrane of the balloon device 59, such that the therapeutic compound or the components of the therapeutic compound contact the clot 90. The inner core 34 can be inserted into the outer sheath 16 before, after or concurrently with the expansion of the balloon 59 and/or the delivery of the therapeutic compound. Similarly, the ultrasound radiating member 40 can be operated before, after, or concurrently with the expansion of the balloon device 59 and/or the delivery of the therapeutic compound.

### Small Vessel Ultrasonic Catheter

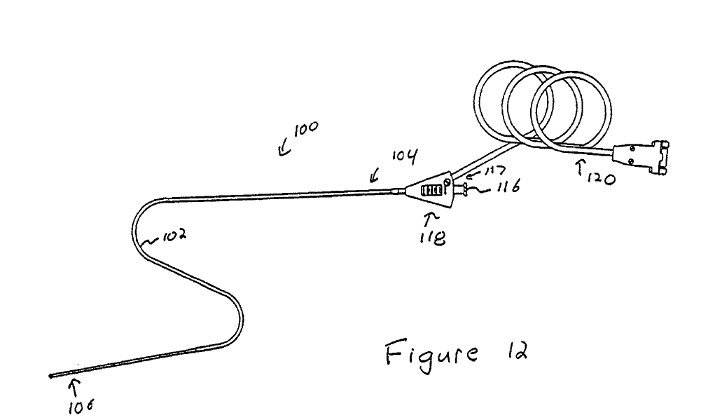

FIGS. 12 through 13B illustrate one embodiment of an ultrasonic small vessel catheter 100. This embodiment is particularly suited for use with small vessels of the distal anatomy, such as, for example, the small neurovascular vessels in the brain.

As illustrated in FIG. 12 and 13A, the ultrasonic small vessel catheter 100 generally comprises a multi-component elongate flexible tubular body 102 having a proximal end 104 and a distal end 106. As with the long segment catheter described above, the tubular body 102 and other components of the small vessel catheter 100 can be manufactured in accordance with any of a variety of techniques well known in the catheter manufacturing field. Additionally, suitable material dimensions can be readily selected based on the natural and anatomical dimensions of the treatment site and of the desired percutaneous access site.

As illustrated in FIG. 13A, in certain embodiments the elongate flexible tubular body 102 further comprises an outer sheath 108 that is positioned over an inner core 110. In such embodiments particularly suited for use in the neurovascular system, the outer sheath 108 comprises extruded PTFE, PEEK, PE, polymides, braided polymides and/or other similar materials. The outer sheath 108 preferably has an outside diameter of approximately 0.039 inches at its proximal end, and approximately 0.033 inches to 0.039 inches at its distal end. In such embodiments, the outer sheath 108 has an axial length of approximately 150 centimeters. In other embodiments, the outer sheath 108 can be formed from a braided tubing formed of, by way of example, high or low density polyethylenes, urethanes, nylons, and so forth. In such embodiments, the tubular body 102 has enhanced flexibility. In modified embodiments, the outer sheath 108 includes a stiffening member (not shown) at the proximal end 104 of the tubular body 102.

Still referring to FIG. 13A, the inner core 110 defines, at least partially, a central lumen 112. The central lumen 112 preferably extends through the length of the small vessel catheter 100, such that a guidewire (not shown) can be passed therethrough. In such embodiments, the central lumen 112 comprises a distal exit port 114 and a proximal access port 116. As illustrated in FIG. 12, the proximal access port 116 is hydraulically connected to the drug inlet port 117 of a backend hub 118. Backend hub 118 is attached to the proximal end 104 of the tubular body 102. The illustrated backend hub 118 is preferably attached to a control box connector 120, the utility of which will be described below.

As described above, the central lumen 112 is preferably configured to receive a guidewire (not shown). In one embodiment, the guidewire has a diameter of approximately 0.010 inches to 0.012 inches. The inner core 110 is preferably formed from polymide or a similar material, which ins certain embodiments is braided to increase the flexibility of the tubular body 102.

Referring now to FIGS. 13A and 13B, the distal end 106 of the tubular body 102 preferably includes an ultrasound radiating member 124. As illustrated, the ultrasound radiating member 124 comprises an ultrasonic transducer, which converts, for example, electrical energy into ultrasonic energy. In a modified embodiment, the ultrasonic energy is generated by an ultrasonic transducer that is remote from the ultrasound radiating member 124 and the ultrasonic energy can be transmitted to the ultrasound radiating member 124 via, for example, a transmission wire.

As illustrated, in certain embodiments, the ultrasound radiating member 124 has the shape of a hollow cylinder. Thus, if the ultrasound radiating member is positioned over the inner core 110, the central lumen 112 will extend through the ultrasound radiating member 124. The ultrasound radiating member 124 can be secured to the inner core 110 in any suitable manner, such as with an adhesive. In other embodiments, the ultrasound radiating member 124 can be of a different shape, such as, for example, a solid rod, a disk, a solid rectangle or a thin block attached to the inner core 110. The ultrasound radiating member 124 can also be formed from a plurality of smaller ultrasound radiating members. The illustrated arrangement is generally preferred because it provides enhanced cooling of the ultrasound radiating member 124. Specifically, as will be explained in more detail below, a therapeutic compound can be passed through the central lumen 112, thereby providing a heat sink for heat generated by the ultrasound radiating member 124.

As mentioned above, suitable frequencies for the ultrasound radiating member 124 include, but are not limited to, from about 20 kHz to about 20 MHz. Preferably, the frequency is between about 500 kHz and 20 MHz and is more preferably between about 1 MHz and 2 MHz. In yet another embodiment, the frequency is about 2 MHz.

As described above, in certain embodiments, ultrasonic energy is generated from electrical power supplied to the ultrasound radiating member 124. For example, the electrical power can be supplied through the control box connector 120, which is connected to first and second wires 126, 128 that extend through the tubular body 102. The first and second wires 126, 128 are preferably secured to the inner core 110, laid along the inner core 110 or positioned freely in the space between the inner core 110 and the outer sheath 108. In the illustrated embodiment, the first wire 126 is connected to the hollow center of the ultrasound radiating member 124, and the second wire 128 is connected to the outer periphery of the ultrasound radiating member 124. In such embodiments, the ultrasound radiating member 124 is preferably formed from, for example, a piezolectic ceramic oscillator or a similar material.

With continued reference to FIGS. 13A and 13B, the distal end 106 of the tubular body 102 preferably comprises a sleeve 130. In such embodiments, the sleeve 130 is generally positioned around the ultrasound radiating member 124. The sleeve 130 is preferably constructed from a material that readily transmits ultrasonic energy. Suitable materials for the sleeve 130 include, but are not limited to, polyolefins, polyimides, polyesters and other low ultrasound impedance materials. Low ultrasound impedance materials are materials which readily transmit ultrasonic energy with minimal absorption. In certain embodiments, the proximal end of the sleeve 130 attaches to the outer sheath 108 with an adhesive 132. In a similar manner, the distal end of the sleeve 130 can be attached to a catheter tip 134. As illustrated, the catheter tip 134 has a generally rounded shape, and is also attached to the distal end of the inner core 110.

In such embodiments, the tubular body 102 is preferably divided into at least three sections of varying stiffness. The first section, which preferably includes the proximal end 104 of the tubular body 102, has a relatively higher stiffness. The second section, which lies between the proximal end 104 and the distal end 106 of the tubular body, has a relatively lower stiffness. This configuration facilitates movement and placement of the small vessel catheter 100 within the neurovascular system. The third section, which preferably includes the ultrasound radiating element 124, is generally stiffer than the second section due to the presence of the ultrasound radiating element 124.

With continued reference to FIG. 13B, the small vessel catheter 100 preferably comprises at least one temperature sensor 136 located at the distal end 106 of the tubular body 102, near the ultrasound radiating member 124. Suitable temperature sensors include, but are not limited to, diodes, thermistors, thermocouples, RTDs and fiber optic temperature sensors that use thermalchromic liquid crystals. As with the long segment catheter described above, the temperature sensors are preferably operatively connected to a control box (not shown) by a control wire. In such embodiments, the control wire preferably extends through the tubular body 102 and backend hub 118, and is operatively connected to a control box through the control box connector 120. The control box preferably includes a feedback control system, such as the control system described above with reference to FIG. 9. As with the long segment catheter described above, the control box is preferably configured to monitor and control the power, voltage, current and/or phase supplied to the ultrasound radiating member 124. In this manner, the temperature of the small vessel catheter 100 can be monitored and controlled.

In use, a free end of a guidewire is percutaneously inserted into the arterial system at an insertion site. The guidewire is then advanced through the vascular system to a treatment site 88 that includes a clot 90. The guidewire wire is then preferably directed through the clot 90.

In such embodiments, the small vessel catheter 100 is then percutaneously inserted at the insertion site and is advanced along the guidewire towards the treatment site 88 using traditional over-the-guidewire techniques. The small vessel catheter 100 is advanced until the distal end 106 of the tubular body 102 is positioned at or within the clot 90. The distal end 106 of the tubular body 102 can include radiopaque markers to aid positioning the distal end 106 of the tubular body 102 within the treatment site 88.

After the distal end 106 of the tubular body 102 is positioned at the treatment site 88, the guidewire can be withdrawn from the central lumen 112. A therapeutic compound source (not shown), such as a syringe with a Luer fitting, is connected to the drug inlet port 117 and the control box connector 120 is connected to the control box. After such connections are made, the therapeutic compound can be delivered through the distal exit port 114 to the clot 90 via the central lumen 112. As with the long segment catheter, suitable drug solutions for treating thrombus include, but are not limited to, an aqueous solution containing Heparin, Uronkinase, Streptokinase, and/or TPA.

Activating the ultrasound radiating member 124 causes ultrasonic energy to be delivered through the distal end 106 of the tubular body 102 to the clot. As described above, suitable frequencies for the ultrasonic energy include, but are not limited to, from about 20 kHz to about 20 MHz. Preferably, the frequency is between about 500 kHz and 20 MHz, and is more preferably between about 1 MHz and 2 MHz. In yet another embodiment, the frequency of the ultrasonic energy is about 2 MHz. The therapeutic compound and ultrasonic energy are preferably applied until the clot 90 is partially or entirely dissolved. Once the clot 90 has been dissolved to the desired degree, the small vessel catheter 100 can be withdrawn from the treatment site 88.

In modified embodiments, the small vessel catheter 100 comprises a cooling system for removing heat generated by the ultrasound radiating member 124. As illustrated in FIG. 13A, in such embodiments, a cooling fluid is passed through cooling fluid lumen 138 to remove thermal energy from the region surrounding the small vessel catheter.

### Catheter with Electrically Conductive Core

As described above, and as schematically illustrated in FIGS. 7A through 9, the electrical connections for providing power to the ultrasound radiating members 40 are preferably provided by wires, although other connection techniques can also be used. Details relating to the various techniques for electrically connecting the ultrasound radiating members 40 will now be discussed in greater detail.

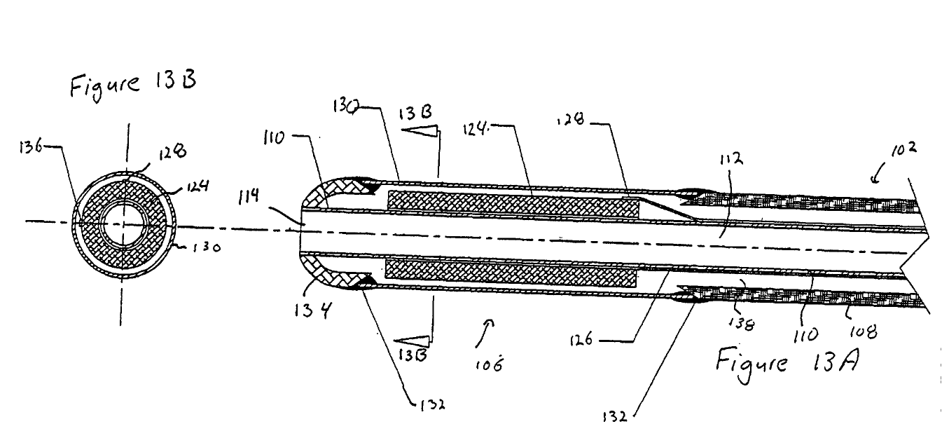

For example, in the preferred embodiments illustrated in FIGS. 14A through 14D, the inner core 34 further comprises an insulating tubing at least partially made from an insulator such as polyimide. In such embodiments, ultrasound radiating members 40, in the shape of hollow cylinders, are placed over the inner core 34, and are situated in the energy delivery section 18. As illustrated, one or more such ultrasound radiating members 40 can be included within the energy delivery section. In such embodiments, a plurality of conductive pathways 210a, 210b (sometimes also referred to as “electrical traces”) are formed in or on the inner core 34 to provide electrical connection to the plurality of ultrasound radiating members 40. The conductive pathways 210a, 210b can be embedded in, etched into or molded on the inner core 34, such that the conductive pathways 210a, 210b are recessed within the inner core 34. In certain embodiments, a layer of additional insulator, such as additional polyimide, is deposited over the conductive pathways 210a, 210b to prevent electrical shorting or other unintended electrical contact with other items. Or, in the absence of such a layer, the conductive pathways 210a, 210b are preferably recessed within the inner core 34 such that they are unlikely to make unintended electrical contact with other items. Proximal connection wires 212 preferably form electrical connection between the conductive pathways 210a, 210b and other electronics outside the energy delivery section 18 of the inner core 34.

The conductive pathways 210a, 210b in the inner core 34 are configured to electrically connect the ultrasound radiating members 40 with each other, and/or with a feedback control system 68. For example, in the embodiment illustrated in FIG. 14B, the ultrasound radiating members 40 comprise ultrasonic transducers comprising a piezoelectric material 214 sandwiched between an outer electrode 216a and an inner electrode 216b. In such embodiments, the conductive pathways 210a, 210b are electrically connected to the electrodes 216a, 216b. Specifically, conductive pathway 210a, which is embedded in the inner core 34, is electrically connected to inner electrode 216b, which is positioned adjacent the inner core 34. Electrical contact is created by soldering the conductive pathway 210a to the inner electrode 216b, thereby forming an electrical contact point 218. In embodiments wherein additional insulator is formed over the conductive pathway 210a, a portion of this insulation is preferably removed to expose the conductive pathway 210a, thereby allowing the inner electrode 216b to be soldered to the conductive pathway 210a. In modified embodiments, a plurality of conductive pathways 210a, 210b are electrically connected to electrodes 216a, 216b, thereby permitting each of the ultrasound radiating members 40 to be activated. In other embodiments, conductive pathways 210a, 210b are configured to allow the ultrasound radiating members 40 to be driven independently.

The various embodiments illustrated in FIGS. 14A and 14B can be fabricated using a wide variety of techniques. For example, in one preferred fabrication method, conductive pathways 210a, 210b are patterned or etched into the inner core 34. In other embodiments, metallization is formed on the inner core 34 and subsequently patterned to create the conductive traces 210a, 210b. Any number of conductive pathways, 210a, 210b can be provided using such techniques. Preferably, however, the inner core 34 comprises at least a sufficient number of conductive pathways 210a, 210b to provide electrical contact to least some of the ultrasound radiating members 40. As described above, in certain embodiments, additional insulating material is deposited on the insulating inner core 34 to prevent unintended electrical contact between the conductive pathways 210a, 210b and other items. In such embodiments, the conductive pathways 210a, 210b are preferably exposed at locations where electrical contact is to be provided. The hollow cylindrical ultrasound radiating members 40 can be slid over the inner core 34, and can be positioned to create electrical contact between the appropriate conductive pathway 210a, 210b and the inner electrodes 216b. Solder disposed adjacent the exposed portion of the conductive trace 210a can be used to form one or more electrical contact points 218. In embodiments in which the ultrasound radiating members are not hollow cylinders, the radiating members may be attached to the inner core 34 such that they are positioned over the space where electrical contact is provided.

As illustrated in FIG. 14B, the inner core 34 preferably includes a central lumen 51 configured for receiving a guidewire or for delivering a therapeutic compound. However, in modified embodiments the central lumen 51 is excluded. As illustrated in FIG. 14C, in certain embodiments a protective jacket 220 covers the ultrasound radiating member 40. In such embodiments, the protective jacket is fixed in place by an epoxy 222 or any other suitable adhesive. As illustrated in FIG. 14D, an outer sleeve 224 can be formed over the inner core 34, the outer sleeve 224 configured to cover the ultrasound radiating member 40 and adjacent regions. In such embodiments, a potting material 226, such as epoxy or any other material that provides flexibility and support to the catheter 10, is be added beneath the outer sleeve 224.

In addition to conductive pathways 210a, 210b, in other embodiments electrical circuitry can be formed directly on or in the inner core 34. Such circuitry can include, for example, a multiplexer configured to allow multiple electronic signals to be provided on a single wire, thereby reducing the total number of wires within the catheter 10.

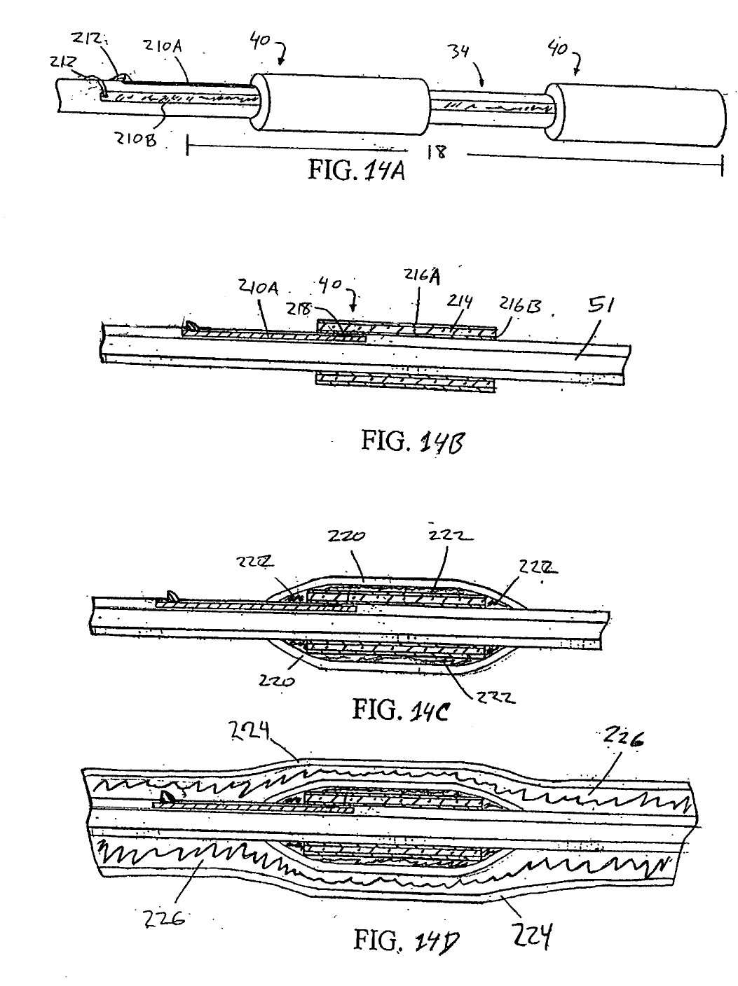

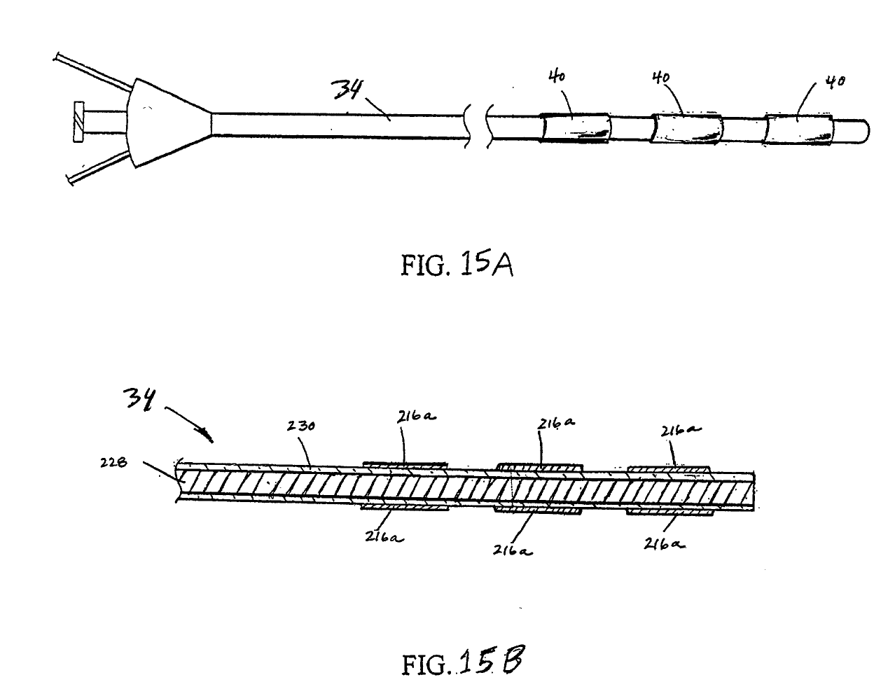

The various embodiments described herein can be modified in various ways to obtain certain design advantages. For example, as illustrated in FIGS. 15A and 15B, in certain embodiments, the inner core 34 comprises conductive material 228 (such as, for example, metal wiring or tubing) having a piezoelectric film 230 deposited thereon. The piezoelectric film 230 can comprise, for example, a piezoelectric polymer. One or more outer electrodes 216a comprising, for example, metal or other conductive material, is formed on the outside surface of the inner core 34 over the piezoelectric film 230. The conductive inner core 34 thus serves as a counterpart inner electrode 216b to the outer electrode 216a, with the piezoelectric firm 230 disposed therebetween. This configuration therefore creates ultrasound radiating members 40 at desired locations. Electrical wiring (not shown) can be connected to the outer electrodes 216a, thereby allowing the individual ultrasound radiating members 40 to be driven independently, if desired. Other forms of electrical connection can also be employed.

Still referring to FIGS. 15A and 15B, in such embodiments, when an oscillating voltage is applied across the piezoelectric file 230, mechanical vibrations can be induced in the piezoelectric film 230, thereby creating ultrasonic energy. In modified embodiments, the inner core 34 comprises non-metallic or non-conducting material with a surface metallic layer formed thereon. The surface metallic layer forms an inner electrode 216b, thereby allowing a voltage to be applied across the piezoelectric film 230. If desired, a single outer electrode 216a can be sued to create a single ultrasound radiating element 40, wherein the inner core 34 conductive material 228 serves as the counterpart inner electrode. As before, this configuration allows a voltages to be applied across the piezoelectric film 230. In other embodiments, a central lumen 51 (not shown) through the inner core 34 is provided to receive a guidewire or deliver a therapeutic compound.

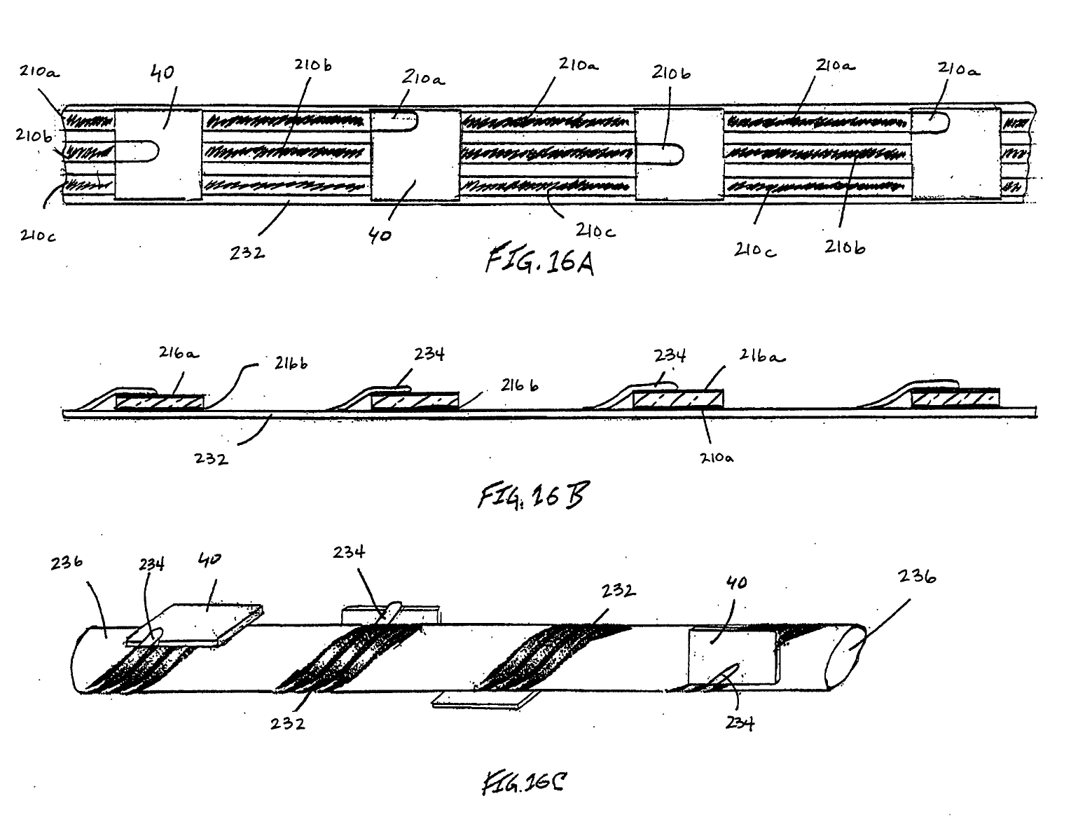

FIGS. 16A through 16D illustrate modified embodiments of an inner core 34 configured to be placed within a catheter 10. In such embodiments, the ultrasound radiating members 40 are mounted on an elongate insulating ribbon strip 232. The elongate insulating ribbon strip 232 comprises a plurality of conductive pathways 210a, 210b, 210c running along the length of the elongate insulating ribbon strip 232. The ultrasonic radiating members 40 are preferably piezoelectric devices comprising piezoelectric material sandwiched between an outer electrode 216a and an inner electrode 216b. The outer and inner electrodes 216a, 216b preferably comprise a metal or any other conductive material. Additionally, the ultrasound radiating members 40 preferably have a planar geometry, and can have any cross-sectional shape when viewed from the top or bottom (that is, as viewed, for example, in FIG. 16C). Preferred cross-sectional shapes for the ultrasound radiating members 40 include, for example, circles and rectangles.

In such embodiments, the conductive pathways 210a, 210b, 210c preferably have an insulating layer formed thereon to prevent unintended electrical contact with other items. As described above, however, portions of the insulating layer can be removed to form electrical contact between the ultrasound radiating members 40 and one or more of the conductive pathways 210a, 210b, 210c.

For example, in the preferred embodiment illustrated in FIGS. 16A through 16D, the ultrasound radiating members 40 are preferably placed on the elongate insulating ribbon strip 232 over a location where a portion of a common conductive pathway 210c is exposed. This configuration allows the inner electrodes 216b to be electrically connected to the common conductive pathway 210c. In such embodiments, the other conductive pathways 210a, 210b are electrically connected to the outer electrode 216a of selected ultrasound radiating members 40. To provide the electrical pathway to the outer electrode 216a, a conductive flap 234 can be extended from the conductive pathways 210a, 210b, such that the conductive flap 234 is in electrical contact with the outer electrode 216a, as illustrated in FIGS. 16B and 16C.

In the preferred embodiment illustrated in FIGS. 16A through 16D, the ultrasound radiating members 40 are arranged in a spiral by wrapping the elongate insulating ribbon strip 232 around a mandrel 236. The mandrel 236 preferably comprises polyimide or any other material that can provide structural support for the elongate insulating ribbon strip 232 and ultrasound radiating members 40. By manipulating the spacing between adjacent ultrasound radiating members 40, the ultrasound radiating members 40 can be oriented in various radial directions from the mandrel 236. For example, FIG. 16C illustrates ultrasound radiating members 40 at the top, sides and bottom of the mandrel 236. Such a configuration directs ultrasonic energy in a plurality of radial directions from the energy delivery section 18 of the catheter 10. Although four ultrasound radiating members 40 are illustrated in FIGS. 16A through 16D, more or fewer can be mounted on the elongate insulating ribbon strip 232. Additionally, the spacing between the ultrasound radiating members 40 can be constant or variable, and is preferably between about 0.25 centimeters and 2 centimeters, although other spacing intervals can be used in other embodiments. The exact spacing characteristics can be determined by the requirements of a particular application.

Referring now to the embodiment illustrated in FIG. 16D, a protective jacket 220 is disposed over the ultrasound radiating members 40. The protective jacket 220 can be disposed, for example, by coating the mandrel 236 and accompanying elongate insulating ribbon strip 232 with a sufficient amount of material. The protective jacket preferably comprises an insulating material, such as, for example, polyimide, high or low density polyethylenes, urethanes, nylons and so forth. In a modified embodiment, the mandrel 236 is removed after disposing the protective jacket 220 over the elongate insulating ribbon strip 232. In such embodiments, a central lumen 51 is thereby formed through the inner core 34. In other embodiments, the mandrel 236 is left in place to provide a solid element without a central lumen. In still other embodiments, the ultrasound radiating members 40 are mounted on an insulating strip that is not a flex circuit, that is, that does not comprise conductive paths. For example, the ultrasound radiating members 40 can be formed on a thin insulating strip, such as polyimide, having electrical conductors disposed therein to provide an electrical connection to the ultrasound radiating members 40.

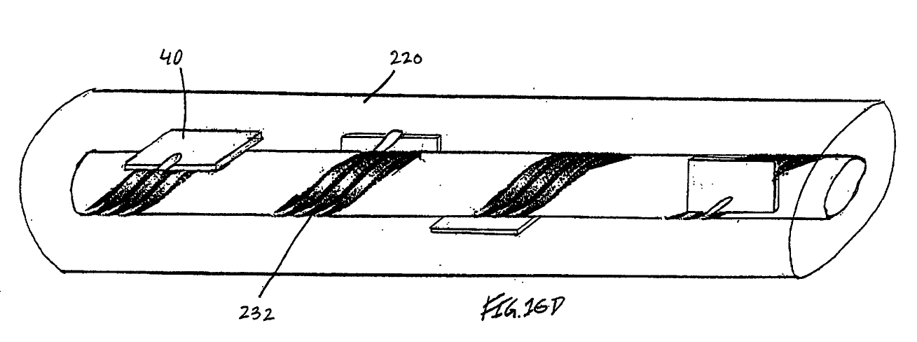

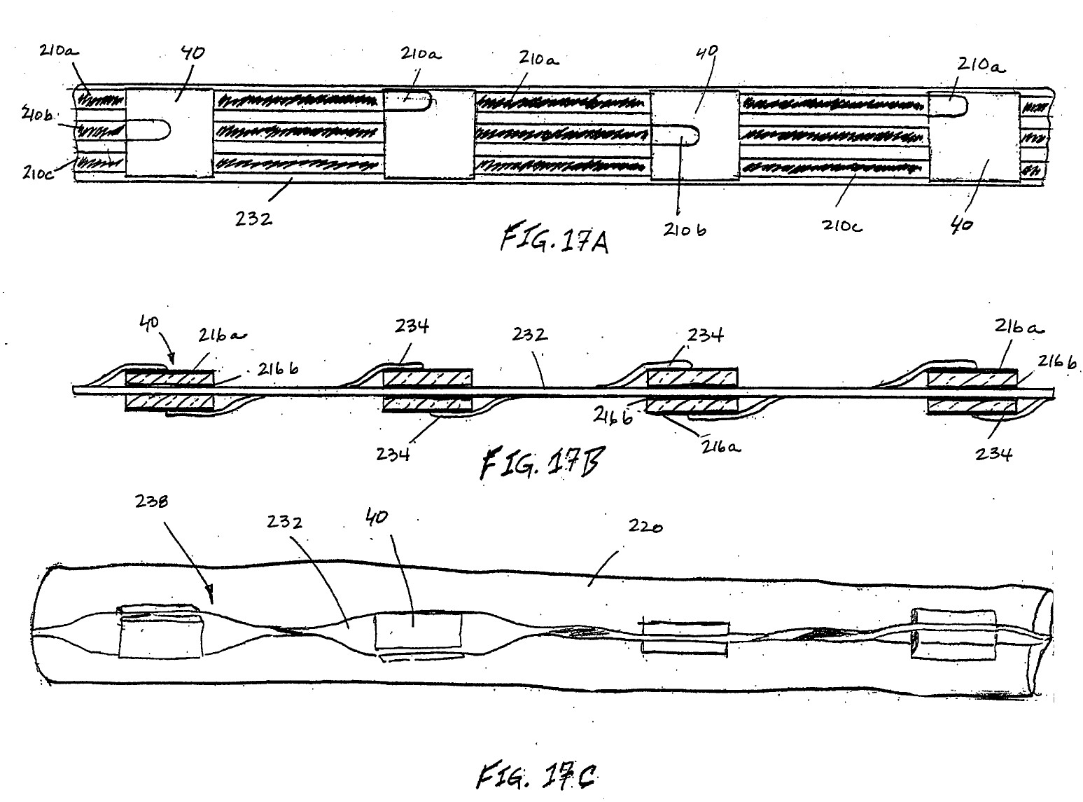

In the preferred embodiment illustrated in FIGS. 17A through 17C, ultrasound radiating members 40 are formed on an elongate insulating ribbon strip 232. The elongate insulating ribbon strip 232 can comprise, for example, a strip of insulating material configured to support a plurality of conductive pathways 210a, 210b, 210c. As illustrated in FIG. 17C, the elongate insulated ribbon strip 232 is twisted to form a helical pattern 238. In such embodiments, the ultrasound radiating members 40 are preferably mounted on opposite sides of the elongate insulating ribbon strip 232, and preferably comprise outer electrodes 216a and inner electrodes 216b. The outer and inner electrodes 216a, 216b preferably have piezoelectric material disposed therebetween, thereby forming ultrasound radiating members 40. In such embodiments, the ultrasound radiating members 40 are preferably positioned over exposed portions of a common conductive pathway 210c such that the inner electrode 216b is electrically connected to the common conductive pathway 210c. Solder or other conducting connective material can be used to complete the electrical connection, thereby forming an electrical contact point between the inner electrode 216b and the common conductive pathway 210c.

The other conductive pathways 210a, 210b can be connected to selected ultrasound radiating members 40 in a manner similar to that described above in association with the embodiments illustrated in FIGS. 16A through 16D. Specifically, to provide an electrical pathway to the outer electrode 216a, a conductive flap 234 can be extended from the conductive pathways 210a, 210b, such that the conductive flap 234 is in electrical contact with the outer electrode 216a, as illustrated in FIG. 117B. In such embodiments, solder or other conductive adhesive or material can be used to form an electrical contact point between the conductive flap 234 and the outer electrode 216a of the ultrasound radiating members 40.

Although ultrasound radiating members 40 are illustrated on both sides of the elongate insulating ribbon strip 232 in FIGS. 17B and 17C, in other embodiments, the ultrasound radiating members 40 are mounted on only one side of the elongate insulating ribbon strip 232, such as illustrated in FIG. 16B. Mounting ultrasound radiating members 40 on opposite sides of the elongate insulating ribbon strip 232, however, allows ultrasonic energy to be directed in opposite radial directions. Additionally, the spacing between the ultrasound radiating members 40 can be constant or variable, and is preferably between about 0.25 centimeters and 2 centimeters, although other spacing intervals can be used in other embodiments. The exact spacing characteristics can be determined by the requirements of a particular application.

By twisting the elongate insulating ribbon strip 232 into a helical shape, as described above, the ultrasound radiating members 40 mounted thereon have varying axial locations and radial orientations. Accordingly, ultrasonic energy can be directed from the catheter 10 in more than one radial direction, and from more than one axial location.

In certain embodiments, as illustrated in FIG. 17C, a protective jacket 220 is formed over the helical pattern 238 that comprises the elongate insulating ribbon strip 232 and the ultrasound radiating members 40. In such embodiments, a potting material is preferably disposed between the protective jacket 220 and the ultrasound radiating members 40. The protective jacket 220 preferably comprises one or more insulating materials, such as polyimides, high or low density polyethylenes, urethanes, nylons, epoxies, silicones, or glues. In such embodiments, the protective jacket 220 is by coating the elongate insulating ribbon strip 232 and the ultrasound radiating members 40 with such a material, thereby providing a smooth, round outer surface on the inner core 34.

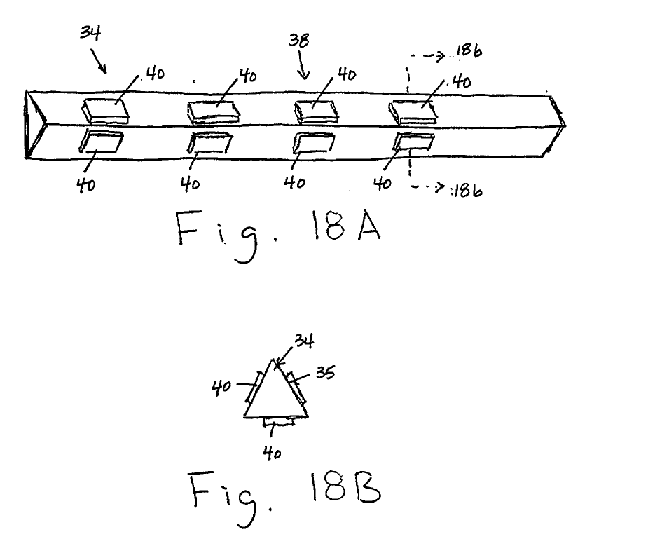

FIGS. 18A and 18B illustrate additional preferred embodiments of an elongate inner core 34 for use with a catheter 10. In this embodiment, a plurality of substantially flat ultrasound radiating members 40 are preferably disposed along the distal end 38 of the inner core 34. In the illustrated embodiment, the inner core 34 has a substantially triangular cross-section. Ultrasound radiating members 40 are preferably disposed along the inner core surfaces 35 in distinct groups. For example, in the embodiment illustrated in FIGS. 18A and 18B, each group comprises three ultrasound radiating members 40 that are disposed around the circumference of the inner core 34. FIG. 18A is a top view of the inner core 34. FIG. 18B is a cross-sectional view of the inner core 34.

Each of the ultrasound radiating members 40 has at least one substantially flat surface capable of being affixed to one of the flat inner core surfaces 35. For example, in one preferred embodiment, the ultrasound radiating members 40 comprise a plurality of flat rectangular lead zirconate titanate (“PZT”) ultrasound transducers that are coupled to the inner core surfaces 35. In such embodiments, the ultrasound radiating members 40 are arranged along the circumference of the inner core 34, such that ultrasonic energy can be delivered in a wide radial field.

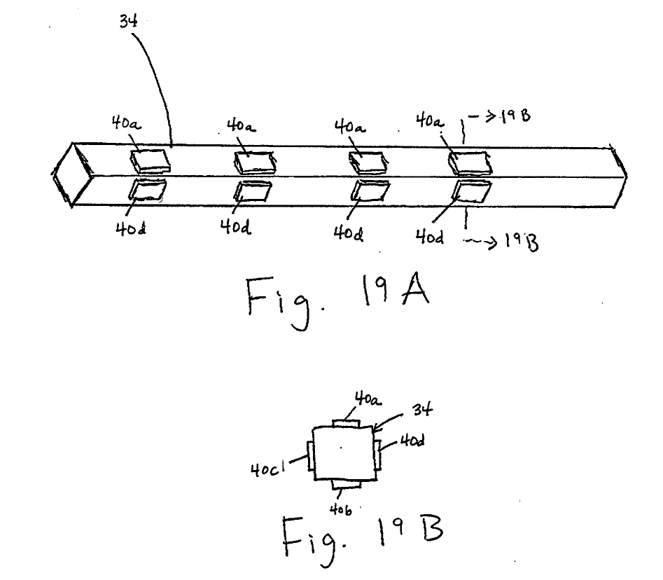

FIG. 19A illustrates a modified embodiment of an elongate inner core 34. In such embodiments, the inner core 34 has a substantially square cross-section. FIG. 19B is a cross-sectional view of the inner core 34 of FIG. 19A. A plurality of ultrasound radiating members 40a-d are mounted along the exterior faces of the inner core 34. The ultrasound radiating members 40a-d are preferably mounted in at least one group of four, more preferably in numerous groups of four. Each of the ultrasound radiating members 40a-d in a particular group is preferably driven by a driving signal that is in phase with the driving signals for the other ultrasound radiating members 40a-d in that group.

In a modified embodiment, two groups of ultrasound radiating members 40a-d are driven out of phase with respect to each other. For example, in the embodiment illustrated in FIG. 19B, ultrasound radiating members 40a and 40b form a first group, while ultrasound radiating members 40c and 40d form a second group. In such embodiments, the first group of ultrasound radiating members 40a, 40b are driven in phase with each other, and the second group of ultrasound radiating members 40c, 40d are driven in phase with each other. However, the first group of ultrasound radiating members 40a, 40b are driven 180° out of phase with respect to the second group of ultrasound radiating members 40c, 40d. In still another embodiment, a larger number of groups of ultrasound radiating members are driven out of phase with respect to each other.

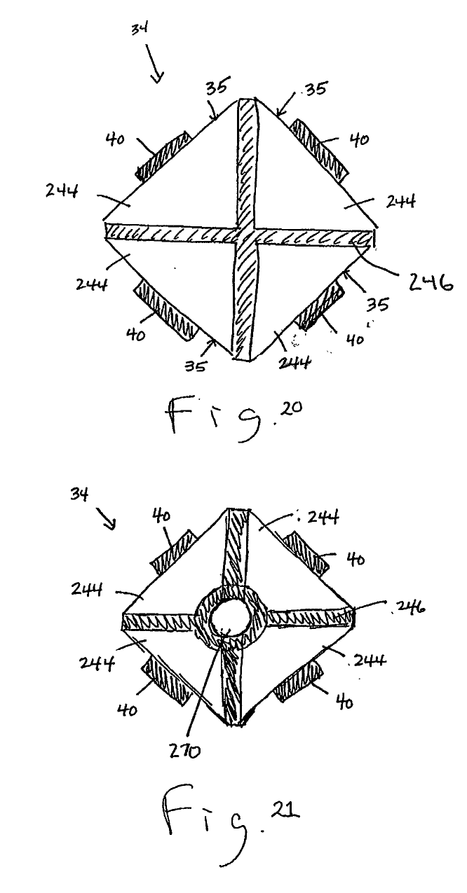

FIG. 20 illustrates a preferred embodiment for supplying electrical power to the ultrasound radiating members 40. In such embodiments, the inner core 34 of an ultrasound catheter preferably comprises four elongate conducting members 244 that extend longitudinally along the length of the inner core 34. Each of the elongate conducting members 244 preferably has a substantially triangular cross-section, such that the four elongate conducting members 244 form a substantially square-shaped cross-section when aligned as illustrated in FIG. 20. In such embodiments, a layer of insulating material 246 is preferably provided between each of the conducting members 244 to electrically isolate the elongate conducting members 244 from each other. When assembled, such embodiments provide a rugged and sturdy inner core 34 having four flat inner core surfaces 35. The four flat inner core surfaces 35 are well-suited for mounting ultrasound radiating members 40 and are each electrically isolated from each other. This composite arrangement also advantageously provides the inner core 34 with increased flexibility. Additionally, such embodiments allow the ultrasound radiating members 40 to be driven independently of each other.

FIG. 21 illustrates another preferred embodiment of an inner core 34 wherein the elongate conducting members 244 and the insulating material 246 are configured to provide a central lumen 270. In such embodiments, the central lumen 270 can be used, for example, to receive a guidewire for facilitating the advancement of a catheter 10 through the patient's vasculature. Or, the central lumen 270 can also be used to transfer therapeutic compounds longitudinally through the catheter inner core 34.

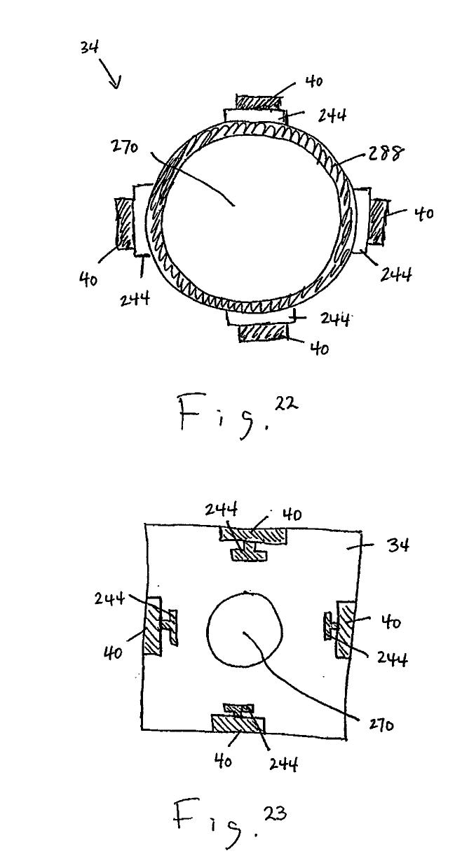

FIG. 22 illustrates yet another preferred embodiment of an inner core 34 comprising a tubular sheath 288, preferably having a circular cross-section. In such embodiments, the tubular sheath 288 defines a central lumen 270 along the longitudinal axis of the inner core 34. Preferably, a plurality of elongate conducting members 244 are disposed along the exterior surface of the tubular sheath 288, and extend longitudinally thereon. As illustrated in FIG. 22, a plurality of ultrasound radiating members 40 are mounted on the elongate conducting members 244 to form one or more groups of four ultrasound radiating members 40. In other embodiments, the groups of ultrasound radiating members 40 can have a greater or smaller number of ultrasound radiating members 40. The ultrasound radiating members 40 in each group are preferably arranged to form a circumferential pattern.

FIG. 23 illustrates yet another preferred embodiment of an inner core 34 comprising a plurality of elongate conducting members 306 and ultrasound radiating members 40, both of which are embedded and integrated into the inner core 34. As illustrated, the inner core 34 preferably comprises a central lumen 270 configured to receive a guidewire or deliver a therapeutic compound along the longitudinal axis of the inner core 34. Such embodiments advantageously reduce the profile of the inner core 34, thereby facilitating the insertion of the inner core 34 into an outer sheath (not shown) during manufacture. Additionally, after inserting the inner core 34 illustrated in FIG. 23 into an outer sheath (not shown), such an inner core 34 readily be moved in relation to the outer sheath (that is, axially) during use. Such movement is desirable in certain applications, such as when it is desired to deliver ultrasonic energy to a large treatment site.

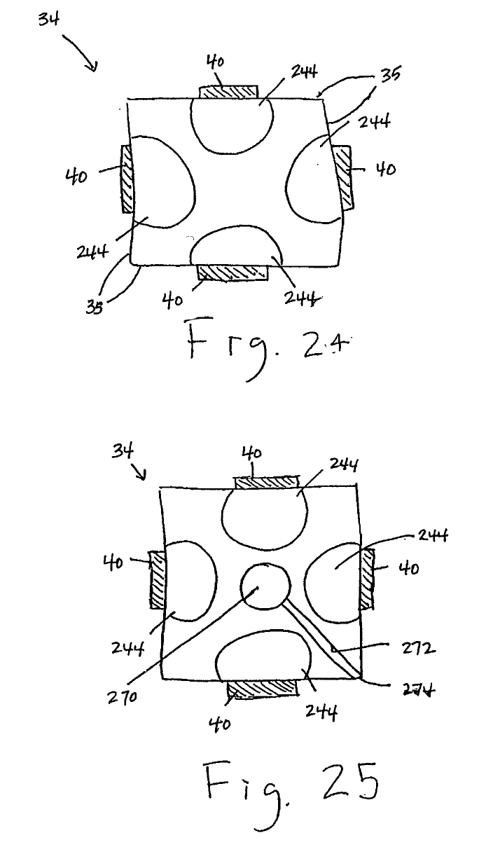

FIG. 24 illustrates yet another preferred embodiment of an inner core 34 wherein a plurality of elongate conducting members 244 are embedded into the inner core 34. As illustrated, in such embodiments a plurality of ultrasound radiating members 40 are preferably mounted along the flat inner core surfaces 35 such that each of the ultrasound radiating members 40 is electrically coupled to an elongate conducting member 244. In embodiments comprising more than four ultrasound radiating members 40 spaced axially along the inner core 34, four outer wires (not shown) preferably extend longitudinally along each side of the inner core 34. The outer wires preferably provide an electrical connection between axially spaced ultrasound radiating members 40.

FIG. 25 illustrates yet another preferred embodiment of an inner core 34 comprising a central lumen 270 that extends longitudinally through the inner core 34. In such embodiments, at least one exit lumen 272 is provided in the inner core 34, thereby allowing the central lumen 270 to pass a therapeutic compound from the inner core 34 to a patient's vasculature. The therapeutic compound exits the inner core 34 at side port 274.

In a preferred embodiment, the various configurations of the inner core 34 described herein are disposed within a tubular body 12, as described above with reference to FIG. 1A. In such embodiments, the inner core 34 can be fixed within the tubular body 12 or can be movable within the tubular body 12. In other embodiments, the inner core 34 is coated with a low ultrasound impedance material, preferably a bio-compatible material, for insulating the electrical components contained within the inner core 34. In such other embodiments, the coating material preferably has a low impedance to ultrasound energy. After coating, the tubular body 12 preferably has a substantially circular cross-sectional configuration with an outer diameter between approximately 0.050 inches and 0.100 inches, more preferably between about 0.060 inches and 0.080 inches, and in yet another embodiment approximately 0.068 inches. Other dimensions for the tubular body 12 may be appropriate based on the requirements of a particular application. In still other embodiments, the inner core 34 is housed inside a length of heat-shrink tubing.

The various configurations of the inner core 34 described herein preferably provide the assembled catheter 10 with sufficient structural integrity, or “pushability,” to allow the catheter 10 to be advanced through a patient's vasculature to a treatment site 88 without buckling or kinking. It is also preferably for the assembled catheter 10 to have the ability to transmit torque. Thus, after the proximal end 14 of the catheter 10 is inserted into a patient, the distal end 15 of the catheter 10 can be rotated into a desired orientation by applying torque to the proximal end 14 of the catheter.

A catheter 10 comprising an inner core 34 configured with multiple ultrasound radiating members 40 as described herein can be advantageously used to emit ultrasonic energy in a circumferential pattern. Thus, such a catheter 10 is particularly well suited for focusing ultrasonic energy along a circumferential region of a treatment site. The particular shape and orientation of the ultrasound elements, as well as the frequency of the input signal from the energy source 70 partially determine the direction and pattern of the emitted ultrasonic energy. Such characteristics of the emitted ultrasonic energy may be selected according to the requirements of a particular application.

### Electrical Connectivity

As described above in reference to FIGS. 20 through 25, in embodiments wherein the inner core 34 comprises one or more elongate conducting members 244, the inner core 34 can function as an electrode for electrically connecting one or more ultrasound radiating members 40 to a energy source 70. In such embodiments, the inner core 34 at least partially comprises an electrically conductive material that electrically contacts one ore more ultrasound radiating members 40 at an inner electrode 216b. One or more wires can be attached to the ultrasound radiating members 40, thereby providing an outer electrode 216a, thus completing the electrical circuit. In one preferred embodiment, the outer electrodes 216a are soldered to the outside faces of the ultrasound radiating members 40.

In certain embodiments, each of the ultrasound radiating members 40 are individually connected to an energy source 70, such that each ultrasound radiating member 40 can be excited individually. In a modified embodiment, the ultrasound radiating members 40 are electrically connected in series or in parallel, as described above with reference to FIGS. 7A through 8. Electrically coupling the ultrasound elements in series or in parallel is particularly advantageous in embodiments wherein the ultrasound radiating members 40 are to be powered in a coupled harmonic mode.

In embodiments wherein the ultrasound radiating members 40 are connected in series, a reduced amount of electrical current is required to produce ultrasonic energy from the ultrasound radiating members 40. In comparison, in embodiments wherein the ultrasound radiating members 40 are connected in parallel, an increased amount of electrical current is required to produce ultrasonic energy from the ultrasound radiating members 40. Therefore, smaller wires can be used to connect the ultrasound radiating members 40 in embodiments wherein the ultrasound radiating members 40 are connected in series. Decreasing the diamter of the wires used to connect the ultrasound radiating members 40 allows the catheter 10 to have increased flexibility. On the other hand, in embodiments wherein the ultrasound radiating members 40 are connected in parallel, one of the ultrasound radiating members 40 can fail without adversely affecting the other ultrasound radiating members 40, thereby providing a more robust system. Additionally, a reduced voltage is required to power the ultrasound radiating members 40 when they are connected in parallel.

As described above, the ultrasound radiating members 40 mounts to the inner core 34 can be grouped. In such embodiments, each group of ultrasound radiating members 40 can be electrically connected independently of the other groups. For example, in a preferred embodiment, each group of ultrasound radiating members 40 consists of the ultrasound radiating members 40 mounted on the inner core 34 at a particular point along the longitudinal axis of the inner core 34. In such embodiments, all the ultrasound radiating members 40 in a particular group operates together as a unit. By dividing the ultrasound elements into separate groups, one or more particular groups of can be driven independently of the other groups. This configuration reduces the peak power demand on the energy source 70. Additionally, under this configuration, an individual group of ultrasound radiating members 40 can be driven to produce circumferential ultrasonic energy emission at a single longitudinal position on the inner core 34. Furthermore, each group of ultrasound radiating members 40 can be driven at a different voltage, such that the emission characteristics of the radiated ultrasonic energy are adapted to meet the requirements for a particular application.

In embodiments wherein a large number of ultrasound radiating member groups are provided, multiple wires are built into a flex circuit. Or, each of the several wires can extend individually along the longitudinal axis of the inner core. In application wherein a the catheter 10 is required to have increased flexibility, the wires are preferably coiled around the inner core 34.

### Ultrasound Radiating Member Characteristics

In certain preferred embodiments, the ultrasound radiating members 40 preferably have a substantially flat shape. However, any ultrasound radiating members 40 having at least one flat surface can be attached to the inner core 34. Additionally, it will be appreciated that the ultrasound radiating members 40 can have any shape configured to fit within the catheter 10. For example, alternative ultrasound element shapes include, but are not limited to, circles or rectangles.

The ultrasound radiating members 40 are preferably constructed from a piezoelectric ceramic, such as, for example, PZT. Piezoelectric ceramics typically comprises a crystalline material, such as, for example, quartz, that changes shape when an electrical current is applied to the material. This change in shape, made oscillatory by a oscillating driving signal, creates ultrasonic sound waves. The ultrasound radiating members 40 preferably emit ultrasonic energy having a frequency in the range of about 40.0 kHz to 15.0 MHz, and more preferably in the range of about 1.0 MHz to 3.0 MHz. The actual frequency can be set based on the requirements of a particular application.

In the embodiments described herein, the ultrasonic energy can be emitted as continuous or pulsed waves, depending on the requirements of a particular application. For example, continuous wave ultrasound radiating member 40 (also known as a “CW” or a “Pedoff” transducer) comprises multiple ultrasound radiating members 40 wherein at least one ultrasound radiating member 40 is always producing ultrasonic energy. In other embodiments, multiple ultrasound radiating members 40 are mounted on the inner core 34 such that they transmit ultrasonic energy intermittently.

The ultrasonic energy can be emitted in waveforms having various shapes, such as, for example, sinusoidal waves, triangle waves, square waves or other wave forms. The average acoustic power is preferably between about 0.01 watts and 300 watts, and is in yet another embodiment about 50.00 watts.

### Temperature Sensor Characteristics

In certain embodiments described above, one or more temperature sensors 20, 136 are positioned along the inner core 34 for monitoring the temperature at the treatment site 88 during ultrasonic energy delivery. However, in other embodiments, the temperature sensors 20, 136 can be positioned elsewhere within the catheter 10. Suitable temperature sensors 20, 136 include, but are not limited to, thermistors, thermocouples, RTDs and fiber optic temperature sensors using thermalchromic liquid crystals. Suitable temperature sensor geometries include, but are not limited to, points, patches, stripes and bands. Using the temperature sensors 20, 136 and the feedback control system 68, the tissue at the treatment site 88 can be maintained at a desired temperature for a selected period of time.

### Delivery of Therapeutic Compounds

Ultrasonic energy can enhance the delivery of a therapeutic compound to, as well as the effect of a therapeutic compound at, a treatment site 88. Consequently, certain embodiments described herein are configured to deliver both ultrasonic energy and therapeutic compounds to a treatment site.

For example, in one embodiment, the tubular body 12 of the catheter 10 comprises a plurality of drug delivery ports 58 in the outer sheath 16. For example, in one embodiment the outer sheath 16 comprises ribbons. In such embodiments, when no ultrasonic energy is being delivered, the ribbons are stationary and thereby form a seal to prevent the therapeutic compound from freely flowing out the drug delivery ports 58. However, when the ultrasound radiating members 40 within the tubular body 12 are activated, the ultrasonic energy causes the ribbons to flutter, thereby opening the drug delivery ports 58, and allowing the therapeutic compound to flow freely from the drug delivery ports 58. Consequently, the therapeutic compound is delivered primarily when the proximally-located ultrasound radiating members 40 are producing ultrasonic energy.

### Fabrication Techniques

In one preferred method of fabricating certain catheters 10 described herein, a central wire is positioned inside a polyimide tube. Elongate conducting members 244 are etched or molded onto the surface of the polyimide tube. A plurality of ultrasound radiating members 40 are then positioned over the polyimide tube, and are soldered onto the device at the desired locations. Wires are then soldered onto the exposed surfaces of the ultrasound radiating members 40, thereby connecting the ultrasound radiating members 40 and forming an electronic circuit. A protective jacket 220 is then placed over each ultrasound radiating member 40 and is epoxied in place. An outer sheath 16 is then placed over the entire assembly, which is potted with a flexible insulating epoxy to provide a catheter 10. Alternatively, a flexible epoxy can be applied to the exterior of the outer sheath 16, the flexible epoxy acting as a conformal layer.

In certain embodiments, the potting over the ultrasound radiating members 40 is preferably optimized for increased transmission of the ultrasound energy. In particular, the potting over the ultrasound radiating members 40 preferably comprises a low ultrasound impedance material. In regions between ultrasound radiating members 40, the wires within the catheter 10 preferably have sufficient flexibility to allow the catheter to be navigated through the tortuous vasculature of a patient's body.

While the foregoing detailed description has described several embodiments of the apparatus and methods of the present invention, it is to be understood that the above description is illustrative only and not limiting of the disclosed invention. It will be appreciated that the specific dimensions of the various catheters and inner cores can differ from those described above, and that the methods described can be used within any biological conduit within a patient's body and remain within the scope of the present invention. Thus, the invention is to be limited only by the claims that follow.

What is claimed is:

1. A catheter for delivering ultrasonic energy and therapeutic compounds, the catheter comprising:
an outer sheath having an energy delivery section;
an inner core positioned within the outer sheath, the inner core positioned at least partially within the energy delivery section;
a first ultrasound radiating member mounted along a portion of the inner core within the energy delivery section,
a plurality of conductive pathways supported by the elongate inner core, at least one of the plurality of conductive pathways being electrically connected to the ultrasound radiating member; and
a drug lumen.

2. The catheter of claim 1, wherein the inner core at least partially comprises an electrically insulating material.

3. The catheter of claim 1, wherein the first ultrasound radiating member has a hollow, cylindrical shape, such that the ultrasound radiating member has an inner surface and an outer surface, the inner and outer surfaces each being electrically connected to one of the plurality of conductive pathways.

4. The catheter of claim 1, wherein the plurality of conductive pathways are formed at least partially within a flexible strip carried by the elongate inner core.

5. The catheter of claim 4, wherein the flexible strip is wrapped around the elongate inner core in a spiral pattern.

6. The catheter of claim 4, wherein the first ultrasound radiating member is attached to the flexible strip such that electrical contact is made between the first ultrasound radiating member and at least one of the electrically conductive pathways on the flexible strip.

7. The catheter of claim 1, wherein the inner core has a polygonal cross-sectional shape, such that the inner core has at least a first, a second and a third substantially flat outer faces.

8. The catheter of claim 7, further comprising a second ultrasound radiating member and a third ultrasound radiating member, and wherein the first ultrasound radiating member is mounted to the first outer face, the second ultrasound radiating member is mounted to the second outer face and the third ultrasound radiating member is mounted to the third outer face.

9. The catheter of claim 8, wherein the inner core further comprises an insulating material and an electrically conductive material that is in electrical contact with at least one of the ultrasound radiating members.

10. The catheter of claim 9, wherein the first, second and third ultrasound radiating members are insulated from each other by the insulating material.

11. The catheter of claim 7, wherein the drug lumen is positioned within the inner core.

12. The catheter of claim 1, further comprising a second ultrasound radiating member that is spaced longitudinally from the first ultrasound radiating member and is also electrically connected to at least one of the conductive pathways.

13. The catheter of claim 1, wherein the drug lumen is positioned within the inner core.

14. An apparatus comprising:
a hollow outer sheath configured to be positioned within a patient's vascular system;
an inner core positioned within into the hollow outer sheath;
a plurality of longitudinally spaced ultrasound radiating members mounted along the inner core;
a plurality of electrically conductive pathways supported by the inner core, at least one of the electrically conductive pathways in contact with at least one of the ultrasound radiating members; and
a drug lumen configured to deliver a therapeutic compound to the patient's vascular system.

15. The apparatus of claim 14, wherein the inner core at least partially comprises an electrically insulating material.

16. The apparatus of claim 14, wherein at least one of the ultrasound radiating members has a hollow, cylindrical shape, such that at least one ultrasound radiating member has an inner surface and an outer surface, the inner and outer surfaces each in contact with one of the plurality of electrically conductive pathways.

17. The apparatus of claim 14, further comprising a flexible strip in which the plurality of electrically conductive pathways are formed.

18. The apparatus of claim 17, wherein the flexible strip is wrapped around the inner core in a spiral pattern.

19. The apparatus of claim 17, wherein at least one of the ultrasound radiating members is attached to the flexible strip such that electrical contact is made between the ultrasound radiating member and at least one of the electrically conductive pathways.

20. The apparatus of claim 14, wherein the inner core has a polygonal cross-sectional shape, such that the inner core has at least three substantially flat faces.

21. The apparatus of claim 20, wherein ultrasound radiating members are mounted on at least three of the flat faces of the inner core.

22. The apparatus of claim 20, wherein the inner core further comprises an insulating material and an electrically conductive material that is in contact with at least one of the ultrasound radiating members.

23. The apparatus of claim 22, wherein ultrasound radiating members on a first face of the inner core are electrically insulated from ultrasound radiating members on a second face of the inner core.

24. The apparatus of claim 20, wherein the inner core further comprises an elongate hollow lumen passing therethrough.

25. The apparatus of claim 14, wherein the inner core further comprises an elongate hollow lumen passing therethrough.
